SEQUENCE LISTING (9 sequences)

SEQ ID NO: 1 - DNA, 19 bp, synthetic construct
  misc_feature 1..19: note = NON-MOE
  source 1..19: mol_type = other DNA
gtaggtaaaaacctaatat

SEQ ID NO: 2 - DNA, 20 bp, synthetic construct
  misc_feature 1..20: note = NON-MOE
  source 1..20: mol_type = other DNA
gttcgtttagagaacagatc

SEQ ID NO: 3 - DNA, 21 bp, synthetic construct
  misc_feature 1..21: note = NON-MOE
  source 1..21: mol_type = other DNA
taaagttcgtttagagaacag

SEQ ID NO: 4 - DNA, 15 bp, synthetic construct
  misc_feature 1..15: note = NON-MOE
  source 1..15: mol_type = other DNA
agccctgtatacgac

SEQ ID NO: 5 - DNA, 19 bp, synthetic construct
  misc_feature 1..19: note = MOE
  source 1..19: mol_type = other DNA
gtaggtaaaaacctaatat

SEQ ID NO: 6 - DNA, 20 bp, synthetic construct
  misc_feature 1..20: note = NON-MOE
  source 1..20: mol_type = other DNA
cgtttagagaacagatctac

SEQ ID NO: 7 - DNA, 20 bp, synthetic construct
  misc_feature 1..20: note = MOE
  source 1..20: mol_type = other DNA
cattgtagatgtcaaaagcc

SEQ ID NO: 8 - DNA, 21 bp, synthetic construct
  misc_feature 1..21: note = NON-MOE
  source 1..21: mol_type = other DNA
ctccctcatggtggcagttga

SEQ ID NO: 9 - DNA, 18 bp, synthetic construct
  misc_feature 1..18: note = NON-MOE
  source 1..18: mol_type = other DNA
cggcatgtctattttgta

(19) United States
(12) Patent Application Publication
(10) Pub. No.: US 2024/0216359 A1
(43) Pub. Date: Jul. 4, 2024

(54) TREATMENT OF NEUROLOGICAL DISORDERS

(71) Applicant: GMP BIOTECHNOLOGY LIMITED, Hong Kong (CN)

(72) Inventor: Vuong TRIEU, Agoura Hills, CA (US)

(21) Appl. No.: 18/600,993

(22) Filed: Mar. 11, 2024

Related U.S. Application Data
(63) Continuation of application No. PCT/US2022/075763, filed on Aug. 31, 2022.
(60) Provisional application No. 63/243,177, filed on Sep. 12, 2021.
(60) Provisional application No. 63/243,175, filed on Sep. 12, 2021.

(51) Int. Cl. A61K 31/485; A61K 9/00; A61K 31/10; A61K 31/7125; A61K 45/06; A61K 47/02; A61K 47/12; A61K 47/18; A61K 47/22; A61P 15/00; A61P 15/10; A61P 25/00; C12N 15/113

(52) U.S. Cl. CPC A61K 31/485; A61K 9/0043; A61K 31/10; A61K 31/7125; A61K 45/06; A61K 47/02; A61K 47/12; A61K 47/183; A61K 47/186; A61K 47/22; A61P 15/00; A61P 15/10; A61P 25/00; C12N 15/1136; C12N 2310/11; C12N 2310/315; C12N 2320/31

(57) ABSTRACT

This invention relates to methods, compositions and uses of medicaments for treating or ameliorating the symptoms of neurological disorders, such as Parkinson's Disease and Alzheimer's Disease. These purposes can be achieved with formulations of agents for inhibiting or suppressing expression of TGF-β, alone or in combination with formulations of agents based on apomorphine.

### SEQUENCE LISTING

This application includes a sequence listing submitted electronically as an ASCII file created on Mar. 8, 2022, named 018988-004US1.xml, which is 12,061 bytes in size.

### BACKGROUND

Parkinson's disease (PD) is the second-most common neurological disorder. In recent years, the nonmotor symptoms of PD have received increasing attention including excessive daytime sleepiness (EDS) and sexual dysfunction. EDS is an inability to maintain wakefulness and alertness during the day which results in periods of irrepressible drowsiness or sleep. EDS is a major health hazard in PD, affecting up to three-fourths of all PD patients. Thus, conventional methods and compositions for treating neurological disorders such as PD symptoms including EDS and sexual dysfunction have significant drawbacks in efficacy and side effects.

Apomorphine is a dopamine receptor agonist and has been used intranasally as an adjunctive medication for Parkinson's disease. See T. van Laar et al., Arch. Neurol, 49: 482-484 (1992). Intranasal delivery of apomorphine for Parkinson's disease is disclosed in U.S. Pat. No. 5,756,483. However, apomorphine was used only for the “off-period” symptoms of Parkinson's disease. Thus, conventional methods and compositions for treating PD have significant drawbacks.

There is an urgent need for compositions and methods for treating PD symptoms including EDS and sexual dysfunction. It would be beneficial if early stages of PD could be treated with an agent such as apomorphine. Further, it would be desirable for later stages of PD to be treated with a combination of an agent such as apomorphine and a TGF-beta inhibitor because it is expected that excessive TGF-beta is building in later stages.

There is a long-desired need for a safe and reliable intranasal formulation for apomorphine-based agents for neurological disorders, which is fast acting with reduced adverse side effects.

There is an urgent need for methods and compositions for inhibiting and/or suppressing TGF-β which provide positive clinical results for treating neurological disorders and related pathologies such as Parkinson's Disease, Alzheimer's Disease, male or female sexual dysfunction, and excessive daytime sleepiness.

### BRIEF SUMMARY

This invention relates to therapeutics for treating or ameliorating symptoms of neurological disorders including Parkinson's Disease. More particularly, this invention discloses compositions and agents based on apomorphine and agents for inhibiting or suppressing expression of TGF-β, which provide improved clinical outcomes for such diseases. This invention provides stable formulations of apomorphine-based agents and anti-TGF-β agents including antisense oligonucleotide compositions, as well as methods of use for neurological disorders including Parkinson's Disease, Alzheimer's Disease, male or female sexual dysfunction, and excessive daytime sleepiness.

This invention provides therapies, compositions and methods for treating or ameliorating symptoms of neurological disorders.

In some embodiments, this invention includes agents and compositions for inhibiting or suppressing TGF-beta to provide improved clinical outcomes for neurological disorders.

In further embodiments, this invention provides stable formulations of anti-TGF-beta agents for various therapies for neurological disorders. Examples of anti-TGF-beta agents include TGF-β inhibitors such as antisense oligonucleotides, artemisinin, pharmaceutically acceptable salts forms, esters, polymorphs or stereoisomers thereof, as well as combinations thereof.

In general, the pathology of neurological disorders is unpredictable, therefore new therapies will rely on clinical studies for distinct patient populations.

In further aspects, this disclosure provides highly stable formulations of anti-TGF-beta agents for therapies for neurological disorders. The stable formulations of this invention provide surprisingly improved clinical results. Stable formulations of agents for suppressing TGF-β can be used to reduce symptoms of neurological disorders to relieve disease action.

Methods and compositions of this invention can be used for inhibiting or suppressing factors in the unpredictable pathology of neurological disorders. In certain embodiments, this disclosure provides methods and compositions for inhibiting the activity of TGF-β and/or suppressing TGF-β related pathologies, which can improve the efficacy for treating or ameliorating the symptoms of neurological disorders.

Compositions and formulations of this disclosure can be used for inhibiting and/or suppressing TGF-β to provide positive clinical results for treating neurological disorders.

In some embodiments, enhanced treatments and formulations for treating have been discovered. For example, improved compositions of this disclosure can be used for treating or ameliorating symptoms of neurological disorders such as Parkinson's Disease and Alzheimer's Disease.

In certain embodiments, apomorphine-based compositions of this invention can be used for treating or ameliorating symptoms of neurological diseases, including Parkinson's Disease and Alzheimer's Disease, such as sexual dysfunction, erectile dysfunction and/or excessive daytime sleepiness. Improved apomorphine-based formulations of this invention can control oxidation to improve purity and potency and reduce side effects. The dose of apomorphine-based agents can be reduced in treating symptoms of neurological diseases, including Parkinson's Disease and Alzheimer's Disease.

In additional embodiments, agents of this invention for inhibiting the activity of TGF-β and/or suppressing TGF-β related pathologies can be used for treating or ameliorating symptoms of neurological disorders including sexual dysfunction and excessive daytime sleepiness (EDS) in neurological disorders such as Parkinson's Disease. Improved TGF-β-suppressing formulations of this invention can counteract increases of TGF-β in Parkinson's Disease pathology to reduce sexual dysfunction and EDS symptoms and improve efficacy of treatment.

In further embodiments, this invention provides therapies for treating a neurological disease or disorder by combining use of an agent for inhibiting or suppressing expression of TGF-β with use of apomorphine, an apomorphine pro-drug, or a pharmaceutically acceptable salt or ester thereof. The combined therapy can be used for symptoms of neurological diseases or disorders, including Parkinson's Disease and Alzheimer's Disease, such as male or female sexual dysfunction and/or excessive daytime sleepiness.

Embodiments of this invention include the following:

A therapeutic composition for treating a neurological disease or disorder comprising a therapeutically effective amount of apomorphine, an apomorphine pro-drug, or a pharmaceutically acceptable salt or ester thereof.

The therapeutic composition above, wherein the neurological disease or disorder is Parkinson's Disease, Alzheimer's Disease, male or female sexual dysfunction, or excessive daytime sleepiness.

The therapeutic composition above, wherein the neurological disease or disorder is early or late Parkinson's Disease.

The therapeutic composition above, wherein the apomorphine is Apomorphine Hydrochloride.

The therapeutic composition above, wherein the composition is suitable for intrathecal injection, infusion, or intranasal use.

The therapeutic composition above, wherein the composition is an intranasal powder formulation.

The therapeutic composition above, wherein the composition is an aqueous or non-aqueous formulation comprising any one or more of a pH buffer, a thickening agent, a humectant, a preservative, and one or more pharmaceutical excipients.

The therapeutic composition above, wherein the composition is an aqueous solution of gels, an aqueous suspension, an aqueous liposomal dispersion, an aqueous emulsion, an aqueous microemulsion, or a combination thereof.

The therapeutic composition above, wherein the composition is an aqueous solution having a drug concentration of 5 mg or 10 mg per mL of solution.

The therapeutic composition above, wherein the composition comprises a buffer selected from acetate, citrate, prolamine, carbonate, phosphate, and combinations thereof.

The therapeutic composition above, wherein the composition comprises a thickening agent selected from methyl cellulose, xanthan gum, carboxymethyl cellulose, hydroxypropyl cellulose, carbomer, polyvinyl alcohol, alginates, acacia, chitosan, and combinations thereof.

The therapeutic composition above, wherein the composition comprises a humectant selected from sorbitol, glycerol, mineral oil, vegetable oil, and combinations thereof.

The therapeutic composition above, wherein the composition comprises a bio-adhesive excipient.

The therapeutic composition above, wherein the composition comprises any one or more of glycerin, glycol, propylene glycol, polyethylene glycol, polyethylene glycol 400, ascorbic acid, sodium ascorbate, edetate disodium, and sodium metabisulfite.

The therapeutic composition above, wherein the apomorphine is dispersed to improve solubility.

The therapeutic composition above, wherein the composition is active within 15 to 60 minutes.

The therapeutic composition above, comprising an intranasal dosage form of 0.5 mg or 1 mg per actuation at 0.1 mL per actuation.

The therapeutic composition above, comprising an intranasal formulation comprising one or more of an antioxidant, an antimicrobial, a chelating agent, a preservative, and combinations thereof.

The therapeutic composition above, comprising an intranasal formulation flushed with oxygen and nitrogen.

The therapeutic composition above, comprising an intranasal formulation with a pH of 3.4.

The therapeutic composition above, comprising a stable intranasal formulation after 3 months at 40° C./60% RH, or 24 months at 25° C./60% RH.

The therapeutic composition above, wherein the composition is pharmaceutically tolerable with reduced adverse or side effects.

A use of a therapeutic composition above for treating or ameliorating the symptoms of a neurological disease or disorder in a human subject.

The use above, wherein the neurological disease or disorder is early or late Parkinson's Disease, Alzheimer's Disease, or male or female sexual dysfunction.

A use of a therapeutic composition above in the preparation of a medicament for treating or ameliorating the symptoms of a neurological disease or disorder in a human subject.

The use above, wherein the neurological disease or disorder is early or late Parkinson's Disease, Alzheimer's Disease, or male or female sexual dysfunction.

A use of a therapeutic composition above for treating or ameliorating the symptoms of a neurological disease or disorder in a human subject, wherein the use of the composition is combined with a standard of care treatment for the disease or disorder.

The use above, wherein the neurological disease or disorder is early or late Parkinson's Disease, Alzheimer's Disease, or male or female sexual dysfunction.

A use of a therapeutic composition above for treating or ameliorating the symptoms of a neurological disease in a human or animal body.

The use above, wherein the neurological disease or disorder is early or late Parkinson's Disease, Alzheimer's Disease, or male or female sexual dysfunction.

A method for treating or ameliorating a symptom of a neurological disease or disorder, the method comprising administering the composition above.

The method above, wherein the neurological disease or disorder is early or late Parkinson's Disease.

The method above, wherein the administration is intranasal.

A therapeutic composition for treating a neurological disease or disorder comprising a therapeutically effective amount of an agent for inhibiting or suppressing expression of TGF-β.

The therapeutic composition above, wherein the neurological disease or disorder is Parkinson's Disease, Alzheimer's Disease, male or female sexual dysfunction, or excessive daytime sleepiness.

The therapeutic composition above, wherein the neurological disease or disorder is early or late Parkinson's Disease.

The therapeutic composition above, comprising any one or more pharmaceutically acceptable excipients selected from diluents, stabilizers, disintegrants and anticaking agents.

The therapeutic composition above, comprising any one or more excipients selected from microcrystalline cellulose, polysorbate 80, crospovidone, croscarmellose sodium, and magnesium stearate.

The therapeutic composition above, wherein the composition is suitable for use by intrathecal injection or infusion.

The therapeutic composition above, wherein the composition is pharmaceutically tolerable with reduced adverse or side effects.

The therapeutic composition above, wherein the agent for inhibiting or suppressing expression of TGF-β is an antisense oligonucleotide or inhibitor specific for TGF-β1, TGF-β2, or TGF-β3.

The therapeutic composition above, wherein the agent for inhibiting or suppressing expression of TGF-β is selected from TGF-β2-specific antisense oligonucleotides SEQ ID NOs: 1-9:

SEQ ID NO: 1
gtaggtaaaa acctaatat,
SEQ ID NO: 2
gttcgtttag agaacagatc,
SEQ ID NO: 3
taaagttcgt ttagagaaca g,
SEQ ID NO: 4
agccctgtat acgac,
SEQ ID NO: 5
gtaggtaaaa acctaatat,
SEQ ID NO: 6
cgtttagaga acagatctac,
SEQ ID NO: 7
cattgtagat gtcaaaagcc,
SEQ ID NO: 8
ctccctcatg gtggcagttg a,
SEQ ID NO: 9
cggcatgtct attttgta,,

chemically-modified variants thereof, an artemisinin extract, and a pharmaceutically-acceptable salt, salt polymorph, ester, or isomer thereof, and any combination thereof.

The therapeutic composition above, wherein the agent for inhibiting or suppressing expression of TGF-β is an artemisinin formulation, comprising 90-95% pure artemisinin extract, or a pharmaceutically-acceptable salt, salt polymorph, ester, or isomer thereof, and one or more pharmaceutically acceptable excipients.

The therapeutic composition above, comprising a carrier comprising sterile water for injection, saline, isotonic saline, or a combination thereof.

The therapeutic composition above, wherein the composition is substantially free of excipients.

The therapeutic composition above, wherein the composition is stable for at least 14 days in carrier at 37° C.

The therapeutic composition above, wherein the composition is reconstituted from a lyophilized powder of the composition.

A use of a therapeutic composition above in the preparation of a medicament for treating or ameliorating the symptoms of a neurological disease or disorder in a human subject.

The use above, wherein the neurological disease or disorder is early or late Parkinson's Disease, Alzheimer's Disease, or male or female sexual dysfunction.

A use of a therapeutic composition above for treating or ameliorating the symptoms of a neurological disease or disorder in a human subject, wherein the use of the composition is combined with a standard of care treatment for the disease or disorder.

The use above, wherein the standard of care comprises any one or more additional medicaments comprising anti-inflammatoires, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

The use above, wherein the neurological disease or disorder is early or late Parkinson's Disease, Alzheimer's Disease, or male or female sexual dysfunction.

A use of a therapeutic composition above for treating or ameliorating the symptoms of a neurological disease in a human or animal body.

The use above, wherein the neurological disease or disorder is early or late Parkinson's Disease, Alzheimer's Disease, or male or female sexual dysfunction.

A method for treating or ameliorating a symptom of a neurological disease or disorder, the method comprising administering the composition above.

The method above, wherein the neurological disease or disorder is early or late Parkinson's Disease.

The method above, wherein the administration is intrathecal injection or infusion.

A therapy for treating a neurological disease or disorder in a subject in need, the therapy comprising a combination of:
a therapeutically effective amount of an agent for inhibiting or suppressing expression of TGF-β; and
a therapeutically effective amount of apomorphine, an apomorphine pro-drug, or a pharmaceutically acceptable salt or ester thereof.

The therapy above, wherein the neurological disease or disorder is Parkinson's Disease, Alzheimer's Disease, male or female sexual dysfunction, or excessive daytime sleepiness.

The therapy above, wherein the neurological disease or disorder is early or late Parkinson's Disease.

The therapy above, wherein the apomorphine is Apomorphine Hydrochloride.

The therapy above, wherein the agent for inhibiting or suppressing expression of TGF-β comprises any one or more pharmaceutically acceptable excipients selected from diluents, stabilizers, disintegrants and anticaking agents.

The therapy above, wherein the agent for inhibiting or suppressing expression of TGF-β comprises any one or more excipients selected from microcrystalline cellulose, polysorbate 80, crospovidone, croscarmellose sodium, and magnesium stearate.

The therapy above, wherein the agent for inhibiting or suppressing expression of TGF-β is administered by intrathecal injection or infusion.

The therapy above, wherein the agent for inhibiting or suppressing expression of TGF-β is an antisense oligonucleotide or inhibitor specific for TGF-β1, TGF-β2, or TGF-β3.

The therapy above, wherein the agent for inhibiting or suppressing expression of TGF-β is selected from TGF-β2-specific antisense oligonucleotides SEQ ID NOs: 1-9:

SEQ ID NO: 1
gtaggtaaaa acctaatat,
SEQ ID NO: 2
gttcgtttag agaacagatc,
SEQ ID NO: 3
taaagttcgt ttagagaaca g,
SEQ ID NO: 4
agccctgtat acgac,
SEQ ID NO: 5
gtaggtaaaa acctaatat,
SEQ ID NO: 6
cgtttagaga acagatctac,
SEQ ID NO: 7
cattgtagat gtcaaaagcc,
SEQ ID NO: 8
ctccctcatg gtggcagttg a,
SEQ ID NO: 9
cggcatgtct attttgta,,

chemically-modified variants thereof, an artemisinin extract, and a pharmaceutically-acceptable salt, salt polymorph, ester, or isomer thereof, and any combination thereof.

The therapy above, wherein the artemisinin is 90-95% pure artemisinin extract, or a pharmaceutically-acceptable salt, salt polymorph, ester, or isomer thereof, and one or more pharmaceutically acceptable excipients.

The therapy above, wherein the agent for inhibiting or suppressing expression of TGF-β comprises a carrier comprising sterile water for injection, saline, isotonic saline, or a combination thereof.

The therapy above, wherein the agent for inhibiting or suppressing expression of TGF-β is substantially free of excipients.

The therapy above, wherein the agent for inhibiting or suppressing expression of TGF-β is administered by intrathecal injection or infusion.

The therapy above, wherein the agent for inhibiting or suppressing expression of TGF-β is pharmaceutically tolerable with reduced adverse or side effects.

The therapy above, wherein the agent for inhibiting or suppressing expression of TGF-β is stable for at least 14 days in carrier at 37° C.

The therapy above, wherein the agent for inhibiting or suppressing expression of TGF-β is reconstituted from a lyophilized powder of the composition.

The therapy above, wherein the therapy comprises use of the agents with a standard of care treatment for the disease or disorder.

The therapy above, wherein the standard of care comprises any one or more additional medicaments comprising anti-inflammatoires, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

The therapy above, wherein the agents are administered concurrently, simultaneously, sequentially, or separately.

The therapy above, wherein the apomorphine ingredient is administered alone in an early stage of the neurological disease or disorder, and wherein both the apomorphine ingredient and the agent for inhibiting or suppressing expression of TGF-β are administered in a later stage of the neurological disease or disorder.

The therapy above, wherein the apomorphine ingredient is administered alone in an early stage of the neurological disease or disorder when the subject does not have an elevated level of TGF-β, and wherein both the apomorphine ingredient and the agent for inhibiting or suppressing expression of TGF-β are administered in a later stage of the neurological disease or disorder when the subject has an elevated level of TGF-β.

### BRIEF DESCRIPTION OF THE DRAWINGS

FIG. 1 shows Uptake of Free and Lipofectin®—Complexed FITC-Labeled OT-101 in A 172 Human Glioma Cells.

FIG. 2 shows Effect of OT-101/AP 12009 Treatment on TGF-β2 Secretion from the Human GBM cell line A-172.

FIG. 3 shows analysis of new compositions which have been discovered for inhibiting TGF-β using bioinformatic structure-based ligand design to identify and measure primary and alternative binding sites of TGF-β1. The results determined two sites for binding activity: Site 1 included residues Phe24-Lys37, and Site 2 included residues Cys7-Gln19.

FIG. 4 shows results for clinical pharmacokinetics of intranasal apomorphine in healthy subjects.

FIG. 5 shows results for evaluation of cerebrospinal fluid (CSF) apomorphine levels following intranasal and sublingual administration.

### DETAILED DESCRIPTION OF THE INVENTION

This invention provides compositions, therapies and methods for treating or ameliorating symptoms of neurological diseases or disorders.

In certain respects, this invention encompasses new formulations of apomorphine-based agents which can be used for treating or ameliorating symptoms of neurological diseases including sexual dysfunction and/or erectile dysfunction. Apomorphine-based formulations of this invention can be improved to control, reduce and prevent oxidation of the formulation to maintain purity and potency and reduce side effects. With the improved apomorphine-based formulations of this invention, the dose range of apomorphine-based agents required for treating symptoms of neurological diseases can be reduced with concurrent benefits of increased efficacy of therapy and reduced side effects.

In some respects, this invention provides improved agents for inhibiting the activity of TGF-β and/or suppressing TGF-β related pathologies which can be used for treating or ameliorating symptoms of neurological disorders. For example, among other things, sexual dysfunction and excessive daytime sleepiness (EDS) can be signs in neurological diseases including Parkinson's Disease and Alzheimer's Disease of increased TGF-activity. The improved TGF-β-suppressing formulations of this invention can counteract such increases of TGF-β in neurological pathologies to reduce symptoms including sexual dysfunction and EDS symptoms and improve efficacy of treatment.

In further respects, this invention involved combination therapies for treating a neurological disease or disorder by combining use of an agent for inhibiting or suppressing expression of TGF-β with use of an apomorphine-based agent. The combined therapy can be used for treating neurological diseases or disorders including Parkinson's Disease and Alzheimer's Disease for symptoms such as male or female sexual dysfunction, and excessive daytime sleepiness.

### Use of Formulations of Anti-TGF-Beta Agents

In some embodiments, this invention includes agents and compositions thereof for inhibiting or suppressing TGF-beta to provide improved clinical outcomes for neurological diseases or disorders. Examples of anti-TGF-beta agents include TGF-β inhibitors such as antisense oligonucleotides, artemisinin, pharmaceutically acceptable salts forms, esters, polymorphs or stereoisomers thereof, as well as combinations thereof.

In general, the pathologies of neurological diseases or disorders are unpredictable, therefore new therapies will require clinical studies for distinct patient populations.

In further aspects, this disclosure provides highly stable formulations of anti-TGF-beta agents for therapies for neurological disorders. The stable formulations of this invention can provide surprisingly improved clinical results. Stable formulations of agents for suppressing TGF-β can be used to reduce symptoms of sexual dysfunction and EDS to improve efficacy of treatment.

Methods and compositions of this invention can be used for inhibiting or suppressing factors in the unpredictable pathology of a neurological disorder. In certain embodiments, this disclosure provides methods and compositions for inhibiting the activity of TGF-β and/or suppressing TGF-β related pathologies, which can improve the efficacy for treating or ameliorating the symptoms of neurological disorders.

### Use of Apomorphine-Based Formulations

In some aspects, this invention provides an intranasal apomorphine formulation for treating or ameliorating symptoms of neurological disorders. An intranasal apomorphine formulation of this invention can reduce side effects of administering apomorphine. Such intranasal apomorphine formulations can lower the effective dose required to achieve treatment or amelioration of symptoms.

In further aspects, this invention provides an intranasal apomorphine formulation which can be used to induce TGF-beta expression and restore normal neuronal health in early stage neurological disorders.

In some embodiments, nasal administration of a dopamine receptor agonist can be used in an amount sufficient for treating or ameliorating symptoms of neurological disorders, including Parkinson's Disease and Alzheimer's Disease, such as sexual dysfunction and/or erectile dysfunction.

Additional embodiments of this invention provide a therapy using an intranasal apomorphine formulation which can be used for treating or ameliorating symptoms in neurological disorders along with standard of care for neurological disorders, such as Parkinson's disease (PD) and Alzheimer's Disease, including male or female sexual dysfunction, anxiety, depression, and dementia. Examples of standard of care for these conditions include melatonin, vasodilators, sildenafil, estrogen, flibanserin, levodopa, carbidopa, safinamide, dopamine agonists, amantadine, anticholinergics, benztropine, MAO-B inhibitors, COMT inhibitors, cholinesterase inhibitors, donepezil, rivastigmine, galantamine, and memantine.

Further embodiments of this invention provide a therapy using an intranasal apomorphine formulation which can be used for treating or ameliorating symptoms in neurological disorders, which can reduce the dose needed in a standard of care treatment for symptoms of neurological disorders, such as Parkinson's disease (PD) and Alzheimer's Disease, including male or female sexual dysfunction, anxiety, depression, and dementia.

In certain embodiments, the active ingredient is apomorphine.

Examples of a dopamine receptor agonist agent include apomorphine, chemically modified equivalents and pharmaceutical salts thereof. Chemically modified equivalents of apomorphine may include a pro-drug. The apomorphine-based agent can be dispersed in an aqueous or non-aqueous formulation.

Nasal delivery of a therapeutic composition can include a buffer to maintain the pH of the dopamine receptor agonist, a pharmaceutically acceptable thickening agent, and a humectant. The therapeutic composition may further include one or more pharmaceutical excipients, or a preservative.

A buffer for intranasal administration may be an acetate, citrate, prolamine, carbonate or phosphate buffer.

Examples of a thickening agent include methyl cellulose, xanthan gum, carboxymethyl cellulose, hydroxypropyl cellulose, carbomer, polyvinyl alcohol, alginates, acacia, chitosans and combinations thereof.

Examples of a humectant include sorbitol, glycerol, mineral oil, vegetable oil and combinations thereof.

In some aspects, a formulation for intranasal administration of a therapeutic composition of this disclosure can include a therapeutically effective amount of a dopamine receptor agonist dispersed in a pH-controlled buffer, a thickening agent, and a humectant.

In further aspects, a formulation for intranasal administration of a therapeutic composition of this disclosure may be tolerable, and without adverse side effects.

This invention can also provide an intranasal dosage unit for treating neurological disorders, including PD, such as male or female sexual dysfunction which is tolerable without adverse side effects. The dosage unit can include an effective amount of a dopamine receptor agonist in combination with an intranasal carrier. Examples of an intranasal carrier include buffers. The pH of a buffer may be adjusted to enhance nasal absorption of the dopamine receptor agonist.

This invention can also provide an intranasal dosage unit for treating male or female sexual dysfunction which is fast acting within about 60 minutes of administration, or about 45 minutes, or about 30 minutes, or about 15 minutes.

The intranasal carrier of the intranasal dosage unit is preferably an aqueous solution. Further, the aqueous solution can be selected from the group including aqueous gels, aqueous suspensions, aqueous liposomal dispersions, aqueous emulsions, aqueous microemulsions and combinations thereof.

In some embodiments, a carrier for an intranasal dosage unit may be a non-aqueous solution. Examples of a non-aqueous solution include non-aqueous gels, non-aqueous suspensions, non-aqueous liposomal dispersions, non-aqueous emulsions and non-aqueous microemulsions and combinations thereof.

In further embodiments, an intranasal carrier of the intranasal dosage unit can be a combination of an aqueous solution and a non-aqueous solution.

In certain embodiments, the carrier of the intranasal dosage unit may be a powder formulation. Examples of a powder formulation include powder mixtures, powder microspheres, coated powder microspheres, liposomal dispersions and combinations thereof. Powder microspheres can be formed from various polysaccharides and celluloses such as starch, methylcellulose, xanthan gum, carboxymethylcellulose, hydroxypropyl cellulose, carbomer, alginate polyvinyl alcohol, acacia, chitosans and combinations thereof.

In additional embodiments, an intranasal dosage unit can also include an excipient having bio-adhesive properties.

In certain embodiments, a buffer for an intranasal dosage unit may have a pH of from about 3 to about 10, or from about 3.5 to 7.0.

In some embodiments, an intranasal dosage unit can include a humectant. Examples of a humectant include soothing agents, membrane conditioners, sweeteners and combinations thereof.

In further embodiments, this invention provides a intranasal composition for treating male or female sexual dysfunction containing a therapeutically effective amount of a dopamine receptor agonist which has been dispersed to increase solubility. The composition may include one or more of a glycol derivative, a sugar alcohol, glycerin, propylene glycol, glycerin, polyethylene glycol 400, ascorbic acid, water, sodium ascorbate, and sodium metabisulfite. A glycol derivative may be propylene glycol or polyethylene glycol. A sugar alcohol may be mannitol or xylitol.

This invention is further directed to various formulations and methods for treating symptoms of neurological disorders, including PD, such as sexual dysfunction. Methods of this disclosure can be used for treating or ameliorating symptoms of male or female sexual dysfunction by intranasal administration of a therapeutically effective amount of a dopamine receptor agonist before, during or after sexual activity. Formulations of this invention can reduce adverse side effects.

Examples of a “dopamine receptor agonist” include Apomorphine and its functional equivalents, such as pharmaceutical salts and chemically modified equivalents thereof, including for example pro-drug forms of apomorphine. For example, apomorphine can exist in a free base form or as an acid addition salt.

In some embodiments, a dopamine receptor agonist can be apomorphine hydrochloride, or other pharmacologically acceptable acid addition salts of apomorphine such as hydrobromide, hydroiodide, bisulfate, phosphate, or acid phosphate salts.

Examples of adverse side effects include effects which are incompatible with the health of the user or which are so unpleasant as to discourage the continued use of the formulation. Examples of adverse side effects include hypotension, nausea, vomiting, impaired vision, diaphoresis and ashen coloring.

Apomorphine which is nasally administered can be active in about 30 to about 45 minutes, or about 15 to about 20 minutes, or less than 15 minutes.

A composition of this disclosure can be administered as a nasal spray, drop, suspension, gel, ointment, cream or powder, or in the form of a nasal sponge.

In some embodiments, a composition of this disclosure can be made viscous by including natural gums, methylcellulose and derivatives, acrylic polymers such as Carbopol, and vinyl polymers such as polyvinylpyrrolidone.

In certain embodiments, a composition of this disclosure may contain excipients known in the art, such as preservatives, surfactants, co-solvents, adhesives, antioxidants, buffers, viscosity enhancing compounds, and compounds to adjust the pH or osmolarity.

In further embodiments, a composition of this disclosure may contain an amount of dopamine receptor agonist adjusted for the age and weight of the patient.

In certain embodiments, the dosage level of a dopamine receptor agonist can be adjusted to be effective for achieving an erection in a patient.

In further embodiments, the dosage level of a dopamine receptor agonist can be adjusted to avoid or reduce adverse side effects to the patient. An acceptable level of adverse side effects can be determined by tolerability of the formulation.

In some embodiments, a level of adverse side effects, for example, nausea and/or vomiting, can be reduced or delayed by nasally delivering a dopamine receptor agonist at a controlled dissolution rate. A controlled dissolution rate may provide circulating serum levels and mid-brain tissue levels of the dopamine receptor agonist sufficient to treat sexual dysfunction without inducing nausea and/or vomiting.

In additional embodiments, for doses of apomorphine above about 2 mg, adverse side effects can be reduced by concurrently administering an agent such as nicotine or lobeline sulfate.

In further embodiments, an apomorphine formulation of this invention can be administered along with antiemetic compounds such as metoclopramide, or a phenothiazine such as chlorpromazine, prochlorperazine, pipamazine, thiethylperazine or oxypendyl hydrochloride, or a serotonin (5-hydroxytryptamine or 5-IIT) agonist such as domperidone or odansetron, or a histamine antagonist such as buclizine hydrochloride, cyclizine hydrochloride or dimenhydrinate, or a parasympathetic depressant such as scopolamine, metopimazine, trimethobenzamide, benzquinamine hydrochloride, or diphenidol hydrochloride.

In certain embodiments, an apomorphine formulation of this invention may be an aqueous solution, a non-aqueous solution, or a combination thereof. Aqueous solutions can include aqueous gels, aqueous suspensions, aqueous liposomal dispersions, aqueous emulsions, aqueous microemulsions and combinations thereof. Non-aqueous solutions may include non-aqueous gels, non-aqueous suspensions, non-aqueous liposomal dispersions, non-aqueous emulsions, non-aqueous microemulsions and combinations thereof.

In additional embodiments, an apomorphine formulation of this invention may contain a buffer to maintain the pH, a pharmaceutically acceptable thickening agent, and/or a humectant. A pH buffer can maintain the dopamine receptor agonist in a non-ionized form. A pH buffer can enhance the absorption of the dopamine receptor agonist across nasal mucosa. Examples of buffers include acetate, citrate, prolamine, carbonate, and phosphate buffers.

Non-aqueous formulations may include buffering agents so that an advantageous pH range can be achieved upon contact with nasal mucosa.

In some embodiments, a dopamine receptor agonist formulation of this invention may have a pH of from about 3.0 to about 10.0, or from about 3.0 to about 7.0.

A dopamine receptor agonist formulation of this invention may contain a pharmaceutically acceptable thickening agent. Examples of thickening agents include methyl cellulose, xanthan gum, carboxymethyl cellulose, hydroxypropyl cellulose, carbomer, polyvinyl alcohol, alginates, acacia, chitosans and combinations thereof. A thickening agent can also be used in a powder formulation.

A dopamine receptor agonist formulation of this invention may include a humectant. A humectant can be used in an amount effective to reduce or prevent drying of the mucus membrane or to prevent irritation thereof. Examples of humectants include sorbitol, mineral oil, vegetable oil, glycerol, soothing agents, membrane conditioners, sweeteners, and combinations thereof.

A dopamine receptor agonist formulation of this invention may include pharmaceutically acceptable excipients and/or preservatives.

Examples of preservatives include benzyl alcohol, parabens, thimerosal, chlorobutanol, and benzalkonium chloride. A preservative may be present in a composition in a concentration of up to about 2% by weight.

As used herein, “administered nasally” or “nasal administration” includes that the dopamine receptor agonist is combined with a suitable delivery system for absorption across the nasal mucosa.

In some embodiments, a dopamine receptor agonist formulation of this invention may include a therapeutically effective amount of a dopamine receptor agonist dispersed in a buffer to maintain the pH of the agonist, a pharmaceutically acceptable thickening agent, and a humectant.

In some embodiments, a dopamine receptor agonist formulation of this invention may be effective for the treatment of a sexual dysfunction, such as impotence and/or erectile dysfunction in a male.

Apomorphine Hydrochloride is a selective dopamine receptor agonist known to be involved in mediation of erection. Apomorphine HCl Nasal Spray can be developed for treatment of symptoms of neurological disorders, including Parkinson Disease (PD) and Alzheimer's Disease, such as male erectile dysfunction, female sexual dysfunction, and other neurological manifestations. The formulation may be an aqueous solution at a drug concentration of 5 mg and 10 mg per mL of solution. The formulation can be packaged in multiple dose glass containers and available in dosage strength of 0.5 mg and 1 mg per actuation (0.1 mL per actuation). Screw-on actuators are available for nasal administration.

Formulation of Apomorphine HCl in a liquid dosage form may be effective. Addition of antioxidants, chelating agent, preservative, lowering pH to 3.4, and displacement of oxygen by nitrogen flushing can be used in an acceptable formulation. A packaging system using a container with minimum headspace can reduce interaction of oxygen in the atmosphere with the product. The closure system with Trifoil® liner can provide satisfactory protection against oxygen transmission. Stability studies of the formulations had shown acceptable stability after 3 months at 40° C./60% RH. The formulations can have acceptable stability at a real time of 24 months at 25° C./60% RH.

A. Drug Substance. Apomorphine Hydrochloride is a USP monographed compound. It is a hemihydrate with a molecular formula of C17H17NO2.½H2O and molecular weight of 312.8. It occurs as white to grayish white crystals. One gram dissolves in 50 ml of water and in about 20 ml of water at 80° C.1. pH of a 1% w/v solution is 4.5 to 5.5. Apomorphine HCl is water soluble.

In aqueous solutions, Apomorphine is oxidized to various derivatives of quinolindione, which are devoid of emetic properties. Oxidized solutions are emerald green in color, but the depth of color is not a reliable indication of the extent of oxidation. The rate of oxidation can be retarded by the addition of dilute hydrochloric acid to adjust the pH to be between 3 and 4, with the addition of sodium metabisulfite, and by making the solution essentially free from dissolved oxygen.

B. Excipients and non-active constituents. A challenge to formulation of Apomorphine HCl in an aqueous form is to control the oxidation of drug substance. Functional excipients can be utilized in formulation development. Anti-oxidants, antimicrobial preservative, chelating agent, co-solvents can be added. pH of the formulation can be lowered to 3.4. In addition, deoxygenation by nitrogen displacement can be done.

Additional excipients can be used as discussed below.

1. Citric Acid and Sodium Citrate: Buffer components. Citric Acid has a pKa1 of 3.128, pKa2 of 4.761, and pKa3 of 6.396 at 25° C.2. Apomorphine HCl is stable at low pH between 3.0 and 4.0 and the formulation can be targeted to pH 3.5. Citric Acid with Sodium Citrate as buffer is effective in the desired pH of the formulation.

2. Propylene Glycol: Cosolvent. Propylene Glycol can be used as a solvent in pharmaceutical preparations. It is generally regarded as a nontoxic material, and may be much less toxic than other glycols. It may also act as a preservative. It may be used in spray solutions to stabilize the droplet size. Propylene Glycol can be used in concentrations of 10-30% in aerosol solutions and in concentrations of 5-80% in topicals. It may have humectant and disinfectant properties. Apomorphine HCl is easily oxidized in aqueous medium. Substituting 7% of water with a non-aqueous solvent can improve stability of the formulation.

3. Glycerin: Cosolvent. Glycerin can be used as a solvent in a pharmaceutical preparation. It may have humectant properties. Substituting 5% of water with a non-aqueous solvent can improve stability of the formulation.

4. Ascorbic Acid: Antioxidant. Apomorphine HCl oxidizes in water. Formulation of an aqueous solution may use antioxidants to stabilize the drug. Ascorbic Acid is a reducing agent and adding a small amount can protect the drug because it is more readily oxidized than the drug. Ascorbic acid can be oxidized before Apomorphine, therefore using Ascorbic acid can retard the rate of oxidation of the drug Apomorphine.

5. Sodium Metabisulfite: Antioxidant. Sodium Metabisulfite can be used as an antioxidant in oral, parenteral, and topical pharmaceutical preparations, in concentrations of 0.1%, or 0.01% to 1%. Formulation of an aqueous solution can use antioxidants to stabilize the drug. Sodium Metabisulfite has a redox potential slightly lower than Apomorphine HCl, therefore adding a small amount may protect the drug because it is more readily oxidized than the drug. Sodium Metabisulfite can be used in acidic medium.

6. Edetate Disodium: Chelating Agent. Edetate salts can be used in pharmaceutical formulations as chelating agent and as antioxidant synergists by sequestering trace amounts of metal ions. Edetates can be used in combination with the antimicrobial preservative Benzalkonium Chloride for synergistic effects.

7. Benzalkonium Chloride: Antimicrobial Preservative. Benzalkonium Chloride is a quarternary ammonium compound which can be used as an antimicrobial preservative. In nasal and otic formulations, a concentration of 0.002 to 0.02% can be used.

8. Sodium Hydroxide/Hydrochloric Acid. Small amounts can be used to adjust pH of the final preparation.

9. Purified Water: Solvent. Apomorphine HCl can be dissolved in water with 12% non-aqueous solvent combination. The nasal preparation can be an aqueous liquid form.

An antioxidant can be used to increase stability of an apomorphine formulation. Ascorbic acid or sodium metabisulfite antioxidants can be used as reducing agents, and have lower redox potentials than apomorphine HCl. Formulations containing ascorbic acid at concentrations of 0.1% and 0.01% as well as 0.1% sodium metabisulfite can be used. An Apomorphine HCl 0.5 mg/0.1 mL formulation with 0.01% ascorbic acid may be unstable and turn black after 7 weeks at 40° C. Apomorphine HCl formulation with 0.1% sodium metabisulfite can be stable. An Apomorphine HCl formulation with 0.1% sodium metabisulfite can remain very light yellow in color after 16 weeks at 40° C. Sodium Metabisulfite may act as an antioxidant in the formulation. A combination of antioxidants may be used.

### Use of Formulations of Anti-TGF-Beta Antisense Agents

Methods of this invention include processes for treating or ameliorating the symptoms of neurological disorders in a patient in need. Such processes can be carried out by preparing a pharmaceutical composition including an agent for inhibiting or suppressing expression of TGF-β, and administering a therapeutically sufficient amount of the composition to the subject.

In some embodiments, this disclosure provides uses of a composition of an agent for inhibiting or suppressing expression of TGF-β for treating or ameliorating the symptoms of neurological disorders in a human or animal.

In further embodiments, this disclosure provides uses of a composition of an agent for inhibiting or suppressing expression of TGF-β in the preparation of a medicament for treating or ameliorating the symptoms of neurological disorders.

In processes or uses of this invention, examples of a neurological disorder include Parkinson's disease, Alzheimer's disease, fibrotic disease, and cancer.

This invention provides methods and formulations for subjects having a neurological disorder who may be hospitalized. The hospitalization of a subject can be due to any one of the following:
WHO COVID-19 Clinical Improvement Ordinal Scale Criteria 3, wherein the subject is hospitalized without oxygen therapy;
WHO COVID-19 Clinical Improvement Ordinal Scale Criteria 4, wherein the subject is hospitalized with oxygen by mask or nasal prongs;
WHO COVID-19 Clinical Improvement Ordinal Scale Criteria 5, wherein the subject is hospitalized with non-invasive mechanical ventilation or high-flow oxygen; and
WHO COVID-19 Clinical Improvement Ordinal Scale Criteria 6, wherein the subject is hospitalized with intubation and mechanical ventilation.

A subject of this disclosure who is hospitalized may have age greater than 60 years and may be hospitalized and presenting at least one medical risk factor selected from:
absolute lymphocyte count ≤1000 cells/mm³;
age ≥60 years;
hypertension;
diabetes;
cardiac failure; and
COPD.

In further embodiments, the processes or uses of this invention can be applied where subjects have age greater than 35 years and are hospitalized and exhibiting low PaO₂ less than 76 or 77 mmHg.

In additional embodiments, the disease can include symptoms of fibrosis or multiorgan fibrosis due to any one of lung failure, cardiac failure, kidney failure, and brain cognitive dysfunction. Any of these may be based on a neurological disorder which may be due to Parkinson's disease, fibrotic disease, or cancer.

In further aspects, the methods and/or uses of this invention can be combined or applied with a standard of care treatment recognized for any of Parkinson's disease, fibrotic disease, or cancer.

In further embodiments, the processes or uses of this invention can achieve surprisingly improved subject symptoms. A subject upon administration or use of a composition of this disclosure may have an improved level of an inflammatory biomarker. Examples of inflammatory markers include C reactive protein, erythrocyte sedimentation rate, procalcitonin level, plasma viscosity, and fibrinogen level.

Examples of agents of this disclosure for inhibiting or suppressing expression of TGF-β include antisense oligonucleotides specific for TGF-β1, TGF-β2, or TGF-β3.

Examples of agents of this disclosure for inhibiting or suppressing expression of TGF-β include TGF-β2-specific antisense oligonucleotides given in SEQ ID NOs: 1-9 herein.

SEQ ID NO: 1
gtaggtaaaa acctaatat.,
SEQ ID NO: 2
gttcgtttag agaacagatc.,
SEQ ID NO: 3
taaagttcgt ttagagaaca g.,
SEQ ID NO: 4
agccctgtat acgac.,
SEQ ID NO: 5
gtaggtaaaa acctaatat.,
SEQ ID NO: 6
cgtttagaga acagatctac.,
SEQ ID NO: 7
cattgtagat gtcaaaagcc.,
SEQ ID NO: 8
ctccctcatg gtggcagttg a.,
SEQ ID NO: 9
cggcatgtct attttgta.,

Antisense oligonucleotides given in SEQ ID NOs: 1-9 herein can be chemically-modified, as known in the art.

Examples of agents of this disclosure for inhibiting or suppressing expression of TGF-β include artemisinin extracts, a pharmaceutically-acceptable salt, salt polymorph, ester, or isomer thereof, and any combination thereof. In some embodiments, this disclosure includes a substantially pure artemisinin having a purity of at least 60%, or 70%, or 80%, or 90%, or 95%.

In certain embodiments, agents of this disclosure for inhibiting or suppressing expression of TGF-β may be prepared from a lyophilized powder of the agent.

More specifically, an agent may be a TGF-β2-specific antisense oligonucleotide selected from SEQ ID NOs: 1-9, and administered or used by continuous intravenous infusion at a dose of 140 mg/m² on Days 1 to 7, or at a dose of 1000 mg/m² on Days 1 to 7, or at a dose of 180 mg/m² on Days 1 to 7, or at a dose of 200 mg/m² on Days 1 to 7.

In some embodiments, an agent may be a TGF-2-specific antisense oligonucleotide selected from SEQ ID NOs: 1-9, and chemically-modified variants thereof, and administered or used by continuous intravenous infusion with a Cmax value of from 2 to 3 μg/mL.

In further embodiments, an agent may be a TGF-β2-specific antisense oligonucleotide selected from SEQ ID NOs: 1-9 and chemically-modified variants thereof, and administered or used by continuous intravenous infusion at a dose of 140 mg/m² on Days 1 to 7, either singly or in combination with artemisinin in any form at a dose of 500 mg per day on Days 1 to 5.

Examples of agents of this disclosure for inhibiting TGF-β include agents for specifically inhibiting TGF-β1, TGF-β2, or TGF-β3.

Embodiments of this invention involving administration or use of a composition of an agent can ameliorate or suppress symptoms due to TGF-β induced proteins.

Embodiments of this invention involving administration or use of a composition of an agent can ameliorate or suppress symptoms due to any of Parkinson's disease, fibrotic disease, or cancer.

The agent for inhibiting or suppressing expression of TGF-β may be an artemisinin formulation, comprising 90-95% pure artemisinin extract, or a pharmaceutically-acceptable salt, salt polymorph, ester, or isomer thereof, and one or more pharmaceutically acceptable excipients. Excipients may comprise any one or more pharmaceutically acceptable excipients selected from diluents, stabilizers, disintegrants and anticaking agents. In some embodiments, the excipients may comprise any one or more of microcrystalline cellulose, polysorbate 80, crospovidone, croscarmellose sodium, and magnesium stearate.

In further embodiments, the agent for inhibiting or suppressing expression of TGF-β can be an artemisinin compound or derivative thereof, or a pharmaceutically-acceptable salt, salt polymorph, ester, or isomer thereof.

As used herein, a derivative encompasses chemical modifications that provide structural analogs of a compound. For example, substituents or substitutions of an alkyl group can provide structural analogs.

Embodiments of this invention include processes or uses wherein the agent for inhibiting or suppressing expression of TGF-β is a compound, or ligand comprising a small molecule or polypeptide, that interacts with Site I of TGF-β comprising Trp30 and/or Site II of TGF-β comprising Arg15, Gln19, and Phe8, or a pharmaceutically-acceptable salt, salt polymorph, ester, or isomer thereof.

In some embodiments, the agent for inhibiting or suppressing expression of TGF-β may be a polypeptide or peptide mimetic of Site I of TGF-β comprising residues Phe24-Lys37 and/or Site II of TGF-β comprising residues Cys7-Gln19, or a pharmaceutically-acceptable salt, salt polymorph, ester, or isomer thereof.

In further embodiments, the agent for inhibiting or suppressing expression of TGF-β may be an antibody or antibody fragment, humanized or non-humanized, with affinity for Site I of TGF-β comprising residues Phe24-Lys37 and/or Site II of TGF-β comprising residues Cys7-Gln19.

In additional embodiments, the agent for inhibiting or suppressing expression of TGF-β may be a compound comprising a sesquiterprene lactone or derivative thereof, or a pharmaceutically-acceptable salt, salt polymorph, ester, or isomer thereof.

In certain embodiments, the agent for inhibiting or suppressing expression of TGF-β may be a compound comprising three isoprenyl groups and one lactone ring, or derivative thereof, or a pharmaceutically-acceptable salt, salt polymorph, ester, or isomer thereof.

In various embodiments, the processes or uses of this invention can achieve surprisingly improved outcomes. A subject upon administration or use of a composition of this disclosure may have reduced or suppressed symptoms due to any of Parkinson's disease, fibrotic disease, or cancer.

In certain embodiments, the processes or uses of this invention can achieve surprisingly improved outcomes. A subject upon administration or use of a composition of this disclosure may have reduced intensive care unit duration.

In further embodiments, the processes or uses of this invention can achieve surprisingly improved outcomes. A subject upon administration or use of a composition of this disclosure may have reduced hospitalization duration.

Embodiments of this invention further include pharmaceutical compositions for inhibiting or suppressing expression of TGF-β, or for inhibiting or suppressing an inflammatory response, or for treating or ameliorating the symptoms of any of Parkinson's disease, fibrotic disease, or cancer in a human or animal. The pharmaceutical compositions may contain a TGF-β inhibitor, artemisinin, pharmaceutically acceptable salts forms, esters, polymorphs or stereoisomers thereof, and any combination thereof, as well as a carrier. The TGF-β inhibitor may be selected from TGF-β2-specific antisense oligonucleotides SEQ ID NOs: 1-9 and chemically-modified variants thereof. The carrier may be sterile water for injection, saline, isotonic saline, or a combination thereof.

Importantly, a composition of this disclosure may be substantially free of excipients. Compositions of this invention which are substantially free of excipients have been found to be surprisingly stable in a carrier. In some embodiments, the composition may be stable for at least 14 days, or at least 21 days, or at least 28 days in a carrier at 37° C. In additional embodiments, a pharmaceutical composition for infusion may contain less than 1% by weight of excipients, or less than 0.5% by weight of excipients, or less than 0.1% by weight of excipients.

Embodiments of this invention further contemplate therapeutic modalities in which a composition of this invention is administered or utilized in combination with a standard of care therapy for the disease.

This invention further provides kits comprising a lyophilized powder in a vial at a content of 250 mg each of one or more TGF-β2-specific antisense oligonucleotides selected from SEQ ID NOs: 1-9.

This invention also provides kits comprising a lyophilized powder in a vial at a content of 500 mg of artemisinin or a derivative thereof, or a compound, or ligand comprising a small molecule or polypeptide, that interacts with Site II of TGF-β comprising Arg15, Gln19, and Phe8, a sesquiterprene lactone or derivative thereof, or a compound comprising three isoprenyl groups and one lactone ring and derivatives thereof, or a pharmaceutically-acceptable salt, salt polymorph, ester, or isomer thereof, or any combination of the foregoing.

### Antisense Oligonucleotides

This invention describes compositions and methods for using TGF-β as a valid target for the treatment of any of Parkinson's disease, fibrotic disease, or cancer.

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA oligonucleotides that inhibit protein production by mediating the catalytic degradation of a target mRNA, or by binding to sites on mRNA needed for translation. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone. ASO antiviral agents may block translational processes either by (i) ribonuclease H (RNAse H) or RNase P mediated cleavage of mRNA or (ii) by sterically (non-bonding) blocking enzymes that are involved in the target gene translation.

Without wishing to be bound by theory, sexual dysfunction may be linked to significantly increased TGF-β in of any of Parkinson's disease, fibrotic disease, or cancer. Blocking TGF-β may inhibit or reduce complications due to fibrosis and its spread. Knockdown of TGF-β gene expression may also improve immune responsiveness.

Use of Apomorphine in Combination with TGF-β Suppression

In additional aspects, this invention provides an intranasal apomorphine formulation which can be used for treating or ameliorating symptoms in late stage or severe neurological disorders.

In some embodiments, this invention provides an apomorphine formulation which can be administered with an ommaya reservoir and catheter for treatment of severe neurological diseases, including Parkinson's Disease and Alzheimer's Disease.

Without wishing to be bound by theory, symptoms of neurological diseases, including Parkinson's disease (PD) and Alzheimer's Disease, such as sexual dysfunction, anxiety, depression, and dementia are neurological disorders linked to the deregulation of TGF-β signaling. The TGF-β family signaling pathways modulate psychiatric disorders. Parkinson's disease affects millions of patients. The clinical symptoms of PD include tremor at rest, rigidity, bradykinesia, postural abnormalities and a freezing phenomenon. Some pathological findings in PD include a loss of nigrostriatal dopaminergic (DA) neurons with a subsequent loss of the neurotransmitter dopamine in the corpus striatum, an area of the brain which is important for the control of movement. The TGF-β signaling pathway controls DA neuron development and survival. In PD, there is loss of DA neurons and loss of striatal dopamine, so that TGF-β affects adult brain function and homeostasis. TGF-β activation aborts degeneration of DA neurons with increased amounts of TGF-β1 and TGF-β2 and in the cerebrospinal fluid of PD patients. Apomorphine can increase TGF beta expression, and its use in late stage patients would need to be supplemented with a TGF-beta inhibitor such as OT-101.

Embodiments of this invention provide an intranasal apomorphine formulation which can be used for treating or ameliorating symptoms in late stage or severe neurological disorders in combination with a TGF-beta inhibitor. This combination therapy can provide a balance of TGF-beta-related effects.

In certain embodiments, an intranasal apomorphine formulation can be used for treating symptoms of neurological disorders, including Parkinson's Disease and Alzheimer's Disease, such as male or female sexual dysfunction, and other neurological disorders in combination with a TGF-beta inhibitor.

Examples of TGF-beta inhibitors include an antisense agent against TGF-beta and artemisinin and its derivatives.

Without wishing to be bound by theory, TGF-beta has a normal physiological level needed to maintain neuronal health. However, increased TGF-beta signaling can result in damage in the brain, and the level of TGF-beta must be modulated, and sometimes suppressed.

Embodiments of this invention provide an intranasal apomorphine formulation which can be used for treating or ameliorating symptoms in early stage neurological disorders. Apomorphine can increase TGF beta expression, and during early stage neurological diseases it is of benefit. At later stages of neurological diseases, use of apomorphine needs to be combined with other agents, at least one that is suppressing TGF-beta. This apomorphine therapy can be combined with therapy using a TGF-beta inhibitor to modulate or suppress the level of TGF-beta.

Further embodiments of this invention provide a TGF-beta inhibitor formulation which can be used for treating or ameliorating symptoms in late stage or severe neurological disorders. Certain TGF-beta inhibitors may decrease TGF beta expression, sometimes by accumulation in a region such as the pineal gland. This TGF-beta inhibitor therapy can be combined with therapy using an intranasal apomorphine formulation to modulate or increase the level of TGF-beta.

Additional embodiments of this invention provide a TGF-beta inhibitor formulation which can be used for treating or ameliorating symptoms in late stage or severe neurological disorders combined with therapy using an intranasal apomorphine formulation, and along with standard of care for any neurological disease, including Parkinson's Disease (PD) and Alzheimer's Disease, such as male or female sexual dysfunction, anxiety, depression, and dementia. Examples of standard of care for these conditions include melatonin, vasodilators, sildenafil, estrogen, flibanserin, levodopa, carbidopa, safinamide, dopamine agonists, amantadine, anticholinergics, benztropine, MAO-B inhibitors, COMT inhibitors, cholinesterase inhibitors, donepezil, rivastigmine, galantamine, and memantine.

Further embodiments of this invention provide a TGF-beta inhibitor formulation which can be used for treating or ameliorating symptoms in neurological disorders combined with therapy using an intranasal apomorphine formulation, which can reduce the dose needed in a standard of care treatment for any neurological disease, including Parkinson's Disease (PD) and Alzheimer's Disease, such as male or female sexual dysfunction, anxiety, depression, and dementia.

Additional embodiments of this invention provide a TGF-beta inhibitor formulation which can be used for treating or ameliorating symptoms in neurological disorders neurological disorders combined with therapy using an intranasal apomorphine formulation, and along with standard of care for any of Parkinson's disease (PD), male or female sexual dysfunction, anxiety, depression, and dementia.

In PD, nonmotor symptoms may precede typical motor features of by several years and play a major role in the deterioration of quality of life of patients. Embodiments of this invention include methods for using an apomorphine singly during early disease to maintain neuronal health, and further in combination with a TGF-beta inhibitor in progressing and severe neurological disease. A bioassay for TGF-beta2 in the spinal cord can be used to determine treatment regimen. A pathological level of TGF-beta can be modulated by the addition of a TGF-beta inhibitor.

Additional embodiments of this invention provide a therapy using an intranasal apomorphine formulation for neurological diseases, including Parkinson's Disease and Alzheimer's Disease, with symptoms such as male or female sexual dysfunction, anxiety, depression, and dementia.

Further embodiments of this invention provide a therapy using an intranasal apomorphine formulation for neurological diseases, including Parkinson's Disease and Alzheimer's Disease, with symptoms such as male or female sexual dysfunction, anxiety, depression, and dementia, combined with a TGF-beta inhibitor in a sequential manner. For example, a TGF-beta inhibitor can be administered. The TGF-beta inhibitor, such as OT-101 (Trabedersen) or artemisinin, can inhibit a TGF-beta surge which may be responsible for brain damage. As the neurological disease progresses to a point that TGF-beta exceeds its physiological level, an Apomorphine formulation can be used along with an anti-TGF agent.

All publications including patents, patent application publications, and non-patent publications referred to in this description, as well as the sequence listing are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

The terms “a,” “an,” “the,” and similar terms describing the invention, and in the claims, are to be construed to include both the singular and the plural.

### Examples

Example 1. Example formulation of Apomorphine Hydrochloride. Dose reproducibility was determined by pump weight using a mechanical actuation station. Six different lots of Apomorphine Nasal Spray and three lots of Pfeiffer nasal actuators (to deliver 0.1 g) were studied. Results showed consistency of delivered weights, which gave 11 good sprays after initial priming. Individual sprays were within 15 percent of the target weight and their mean weight was within 10 percent of the target weight.

An example of a formulation is shown in Table 1.

TABLE 1
Example formulation of Apomorphine Hydrochloride

| | | 0.5 mg/0.1 mL | 1.0 mg/0.1 mL |
|---|---|---|---|
| | | Quantity | Quantity |
| # | Ingredients | (% w/w) | (% w/w) |
| 1 | Apomorphine Hydrochloride, USP | 0.50 | 1.0 |
| 2 | Citric Acid Anhydrous, USP | 0.72 | 0.69 |
| 3 | Sodium Citrate Dihydrate, USP | 0.37 | 0.42 |
| 4 | Propylene Glycol, USP | 7.00 | 7.00 |
| 5 | Glycerin, USP (96%) | 4.98 | 4.98 |
| 6 | L-Ascorbic Acid, USP | 0.012 | 0.012 |
| 7 | Sodium Metabisulfite, NF | 0.088 | 0.088 |
| 8 | Edetate Disodium, USP | 0.020 | 0.020 |
| 9 | Benzalkonium Chloride 50% | 0.040 | 0.040 |
| | solution, NF | | |
| 10 | Sodium Hydroxide, NF (1N) | TAP* | TAP* |
| 11 | Hydrochloric Acid, NF diluted | TAP* | TAP* |
| | (10%) | | |
| 12 | Purified Water, USP, to q.s. | 100.0 | 100.0 |

*TAP = To adjust pH

### Example 2. Example of Excessive Daytime Sleepiness in Neurological Diseases Such as Parkinson's Disease and Alzheimer's Disease

Parkinson's Disease (PD) and Alzheimer's Disease have nonmotor symptoms including excessive daytime sleepiness (EDS). EDS is defined as an inability to maintain wakefulness and alertness during the major waking episodes of the day that results in periods of irrepressible need for sleep or unintended lapses into drowsiness or sleep. EDS is a major health hazard in PD, affecting 21-76% of PD patients. EDS in PD is not persistent, and its presence may fluctuate over time. In general, the proportion of PD patients with EDS increases over time with longer follow-up. EDS is associated with and influences other motor and nonmotor symptoms of PD.

P001 was a completed Phase I/II dose escalation study. Primary objective was the determination of the MTD as well as the DLT of 2 cycles as core treatment and up to 8 optional extension cycles of trabedersen (OT-101) administered i.v. for 4 or 7 d every other week, as described in the following. The study followed a classical cohort design with 3 evaluable patients per cohort. Patients treated with the 1st schedule received OT-101 continuously for 7 d, followed by a treatment-free interval of 7 d for each treatment cycle (7-d-on, 7-d-off). After the MTD had been reached for this schedule, a 2nd schedule of 4 d OT-101 administration, followed by a treatment-free interval of 10 d for each treatment cycle was started (4-d-on, 10-d-off). In this treatment schedule the MTD has not been reached.

Insomnia was evaluated in P001. Consistent with the role of TGF-beta in neuronal health, it was found that treatment with OT-101 impacted frequent insomnia in these patients. As such it would be beneficial to PD and Alzheimer's patients who during late stage of the disease were suffering from excessive sleepiness.

Of the 61 pts treated with OT-101, psychiatric changes were observed in 23% of pts with sleeping disorders (13%), insomnia (8%), anxiety (2%) and mood alterations (2%)

Example 3. TGF-β2-specific phosphorothioate antisense oligodeoxynucleotide (OT-101; AP 12009; Trabedersen) is intended to reduce the level of TGF-β2 protein in malignant gliomas. Human TGF-β2-specific phosphorothioate antisense oligodeoxynucleotide (OT-101; AP 12009; Trabedersen), hereafter referred to as OT-101, is intended to reduce the level of TGF-β2 protein in malignant gliomas, and thereby delay the progression of disease.

Antisense oligodeoxynucleotides are short strings of DNA that are designed to downregulate gene expression by interfering with the translation of a specific encoded protein at the mRNA level. OT-101 is a synthetic 18-mer phosphorothioate oligodeoxynucleotide (S-ODN) where all 3′-5′ linkages are modified to phosphorothioates. The molecular formula is C₁₇₇H₂₀₈N₆₀Na₁₇O₉₄P₁₇S₁₇ and the molecular weight 6,143 g/mol. OT-101 was designed to be complementary to a specific sequence of human TGF-β2 mRNA following expression of the gene.

OT-101 is currently supplied as a lyophilized powder in 50-mL glass vials in three different quantities. Each vial is identified by the name of the investigational product, trial number, dosing group, mode of application, quantity of OT-101 contained (in mg), total volume after dissolving (in mL) and resulting concentration (in μM), name of sponsor, name of manufacturer, batch number, vial number, storage temperature, and expiry date. Oncotelic Inc. provides the study medication in closed units, packaged separately for each concentration. The packages contain the appropriate vial(s) and all necessary components of the application system (i.e., syringes, tube, and filter). OT-101 lyophilized powder is dissolved in isotonic (0.9%) aqueous sodium chloride prior to use. A leaflet is enclosed in the packaging with instructions on how to prepare the product for administration of the desired concentration.

### Nonclinical In Vitro Studies of OT-101.

Functional in vitro assays showed that:

OT-101 exhibits an efficient time-dependent uptake into human tumor cells in the presence as well as in the absence of the carrier liposome Lipofectin®.

OT-101 reduces the TGF-β2 secretion by human tumor cells without the use of any carrier.

At the clinically used OT-101 concentrations up to 80 μM over 7 days in A 172 human high-grade glioma cells, 10 μM was the most effective concentration for inhibition of the TGF-β2 production.

OT-101 reduces proliferation of human tumor cells while at the same time stimulating PBMC proliferation. OT-101 does not affect viability of human PBMCs.

OT-101 restores immune function of human PBMC derived from high grade glioma patients demonstrated by immune cell-mediated cytotoxicity assay.

OT-101 inhibits human tumor cell migration.

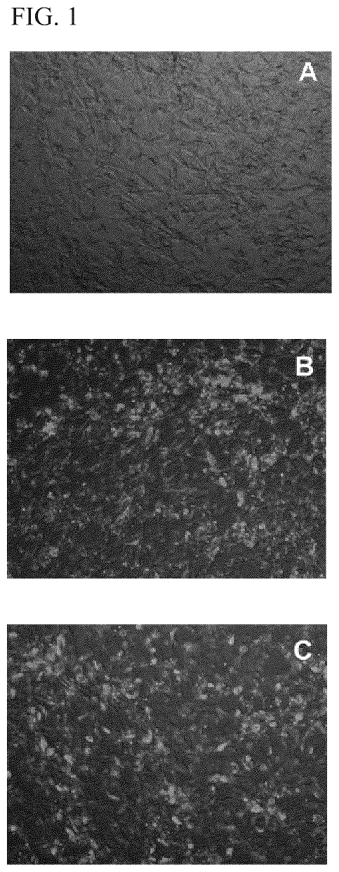

FIG. 1 shows Uptake of Free and Lipofectin®—Complexed FITC-Labeled OT-101 in A 172 Human Glioma Cells. Representative fluorescent micrographs of A-172 human glioma cells after incubation with different preparations are shown: (A) start, 0 h incubation; (B) “naked” FITC-OT-101 (5 μM) without carrier, 48 h incubation; (C) FITC-trabedersen (200 nM) complexed with Lipofectin® (3 μg/mL), 48 h incubation. Referring to FIG. 1, the fluorescent signal increased up to 48 h in human A-172 glioblastoma cells both incubated with FITC-OT-101 with or without Lipofectin®. Uptake of FITC-OT-101 was observed already after 3 h incubation time with and without Lipofectin®. After 48 h the fluorescent signal was detectable in almost all cells and was comparable in intensity in cell preparations incubated with or without Lipofectin®.

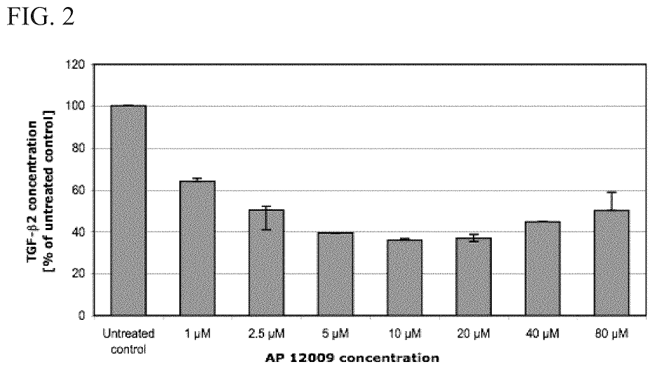

FIG. 2 shows Effect of OT-101/AP 12009 Treatment on TGF-β2 Secretion from the Human GBM cell line A-172. Cells were incubated with the indicated different concentrations of OT-101/AP 12009 (1 μM to 80 μM) for 7 days. Secreted TGF-β2 was measured in cell supernatants by ELISA. Results represent median, minimum, and maximum values from 3 independent experiments.

Effects of OT-101 on TGF-β2 Secretion from primary human high-grade glioma cells. The ability of OT-101 to reduce TGF-β2 secretion by primary human glioma cells was determined by measuring the TGF-β2 concentration in cell culture supernatants using an enzyme-linked immunosorbent assay (ELISA). Glioma cells from 10 high-grade glioma patients were cultured for 72 h (HTZ-209. - 220, -243, -262, -349, -361, -378, -381) or 96 h (A-172) in the presence and absence of OT-101 (5 or 10 μM). In 8 of the 10 glioma cell cultures, the TGF-β2 secretion was reduced by up to 87%.

OT-101-mediated inhibition of human high-grade glioma cell proliferation. Two human HGG cell cultures (HTZ-243 and HTZ-349, representing WHO grade III and IV) were incubated with OT-101 (1 μM to 10 μM). The results in Table 2 showed a concentration- and time-dependent reduction of cell numbers within 6 days.

TABLE 2
Effect of OT-101 on Human High-Grade Glioma Cell Proliferation

| Human | OT-101/AP | Cell number [% of | | |
|---|---|---|---|---|
| glioma cell | 12009 concentration | cells plated] on Day | | |
| line | [μM] | 0 | 3 | 6 |
| HTZ-243 | Untreated | 100 | 105 | 110 |
| | 1 | 100 | 98 | 85 |
| | 5 | 100 | 85 | 55 |
| | 10 | 100 | 88 | 48 |
| HTZ-349 | Untreated | 100 | 109 | 122 |
| | 1 | 100 | 96 | 84 |
| | 5 | 100 | 93 | 47 |
| | 10 | 100 | 94 | 50 |

Two human glioma cell cultures (HTZ-243 and HTZ-349) were treated with OT-101 (1, 5 or 10 μM). Cell number (in % of cell number at start of the experiment) was measured with a hemacytometer. Data show the means of duplicate assessment.

Example 4. Preparation of stable drug agent solutions free of excipients for suppressing TGF-β. This example demonstrates preparation of a stable and compatible solutions of antisense agents for suppressing and inhibiting TGF-β that are substantially free of excipients.

Experiments set forth below showed that a OT-101 solution of 10u M (61.43 μg/mL) in NaCl at 5° C. and 37° C. was surprisingly stable for at least two weeks. Further, OT-101 solutions of 7.35 mg/mL and 25 mg/mL in isotonic saline at 5° C. and 37° C. were surprisingly stable for at least two weeks.

In-use conditions mimicking clinical studies and the outcomes of the studies are shown in Table 3 and Table 4. Table 3 shows results for an antisense oligonucleotide against TGF-β for administration to patients by IV infusion.

TABLE 3
In-use stability study of antisense oligonucleotide against TGF-β

| In-use Conditions | Outcome of the Study |
|---|---|
| Drug solution: 10 μM (61.43 μg/mL) OT-101 in | The Drug Delivery System used in |
| isotonic saline | Clinical Study AP 12009-G005 |
| Flow rate: 4 μL/min (corresponds to 5.76 mL/day) | was suitable for its intended use |
| Storage of the pump (including drug reservoir) and | with regard to the compatibility |
| the non implanted parts of the drug delivery system at | with a 10 μM AP 12009 Drug |
| ambient temperature | Solution |
| Storage of the implanted parts of the drug delivery | |
| system at 37° C. | |
| Drug reservoir content: 50 mL | |
| Duration of test: 8 days | |
| The conditions are same as above (QC-AP0132R) except the | Based on the in-use study results, |
| external component of the Drug Delivery System kept at | the Drug Delivery System for |
| 30° C. to mimic the clincally relevant Climatic Zone III/IV | Clinical Study AP12009-G005 was |
| | considered suitable for its intended |
| | use in Climatic Zone III/IV |
| Drug Conc.: 10 μM (61.43 μg/mL). | Based on the results AP 12009 |
| Temp. 5° C. and 37° C., Diluent 0.9% NaCl, Container | (OT-101) of 10 μM in NaCl at 5° C. |
| 6R Sample Vials, Duration 2 weeks | and 37° C. and AP 12009 (OT-101) |
| Drug Conc.: 1 mg/mL | solution in WFI at 5° C. are stable |
| Temp. 5° C., Diluent WFI, Container 6R Sample | for at least two weeks |
| Vials, Duration 2 weeks | |
| Drug solution: 15 mg/mL (calculated based on a mean | None of the components tested had |
| dosage of 195 mg/m²/d and mean body surface of | impact on the quality of the |
| 1.85 m²) | delivered Drug Solution under in- |
| Flow rate: 1 mL/h (corresponds to 24 mL/day) | use conditions. Based on the result |
| Storage of the pump (including drug reservoir) and | all Drug Delivery Systems |
| the non implanted parts of the drug delivery system at | composed of any combination of |
| 30° C. | one of the tested pumps are |
| Storage of the implanted parts of the drug delivery | considered suitable with regard to |
| system at 37° C. | their compatibility with OT-10 |
| Drug reservoir content: 120 mL | Drug solution |
| Duration of test: 5 days | |

The experiments of Table 3 show that antisense oligonucleotide OT-101 at 10 μM in NaCl at 5° C. and 37° C. was surprisingly stable for at least two weeks. The experiments of Table 3 show that antisense oligonucleotide OT-101 in WFI at 5° C. was stable for at least two weeks.

Table 4 shows results for an antisense oligonucleotide against TGF-β for administration to patients by IV infusion.

TABLE 4
In-use stability study of TGF-β inhibitor trabedersen

| In-use Conditions | Outcome of the Study |
|---|---|
| Drug Conc.: 7.35 mg/mL | Based on the results from the study |
| and 25 mg/mL | concentrated trabedersen (AP |
| Temp. 5° C. and 37° C. | 12009) solutions in NaCl was |
| Diluent 0.9% NaCl | stable for at least two weeks |
| Container 6R Sample Vials, | |
| Duration 2 weeks | |
| Drug Conc.: 18.23 mg/mL | The results of this study |
| Temp. 20-25° C. | demonstrate that the Cadd |
| Diluent 0.9% NaCl | Medication Cassette Reservoir |
| Container: Cadd Medication | warrants sterility of a sterile filled |
| Cassette Reservoir | drug solution for a period of at least |
| Duration: 7 days | 7 days. |

The experiments of Table 4 show that TGF-β antisense oligonucleotide trabedersen at 7.35 mg/mL and 25 mg/mL in NaCl at 5° C. and 37° C. was surprisingly stable for at least two weeks.

A further in-use stability study of OT-101 at 10 UM (61.43 μg/mL) was performed. An analytical stock solution of concentration 1.0 mg/mL and 10 UM (61.43 μg/mL) was used. A 10 μM (61.43 μg/mL) OT-101 clinically relevant concentration in 0.9% NaCl was checked for stability after storage at 5° C. and 37° C., and a 1 mg/mL OT-101 analytical stock solution in Water for Injection was checked for stability after storage at 5° C. for two weeks. The materials used for the experiments are shown in Table 5.

TABLE 5
Materials and drug solution

| Description | Manufacturer | Ref. No. | Lot. No. |
|---|---|---|---|
| OT-101 Working | Avecia, USA | — | AQX-05L-002 |
| Standard | | | Anal A 01/01 |
| 0.9% NaCL | Braun, Germany | 3820084 | 9152A91 |
| solution | | | |
| Ampuwa Water for | Fresenius | 40676.00.00 | 14DD1005 |
| Injection | | | |
| 6R Sample Vials | PharMediPack, | 05000613100 | 20081216 |
| | Germany | | |
| Fluorotec Stoppers | West Phar., USA | 12414110/40 | 1092007924 |
| | | grey | |
| Alu Caps | PharMediPack, | 03500103000 | 0507010302 |
| | Germany | | |

The impurity profiles of the samples were determined by RP-HPLC and the concentrations were determined by UV-spectrometry. The impurities profile of the samples by RP-HPLC are shown in Table 6.

TABLE 6
Reverse Phase HPLC Samples for 10 μM (61.43 μg/mL) Solutions

| | AP | 3′N- | 3′N- | 5′N | | Total Other |
|---|---|---|---|---|---|---|
| | 12009 | 2* | 1' | 1/PO* | CNET* | Imp |
| Acceptance Criteria | ≥85 | ≤0.6 | ≤3.4 | ≤7.6 | ≤5.2 | Report |
| (AP 12009 Drug Product) | | | | | | |
| Description of Sample | | | | | | |
| 10 μM in saline, t = | 96.61 | n.d. | 0.96 | 1.36 | 0.40 | 0.70 |
| 0 d −20° C. | | | | | | |
| 10 μM in saline, t = 7 d 5° C. | 96.76 | n.d. | 0.93 | 1.30 | 0.38 | 0.64 |
| 10 μM in saline, t = 14 d 5° C. | 96.61 | n.d. | 1.00 | 1.37 | 0.37 | 0.67 |
| 10 μM in saline, t = 7 d 37° C. | 96.29 | n.d. | 1.08 | 1.55 | 0.36 | 0.73 |
| 10 μM in saline, t = | 96.09 | n.d. | 0.99 | 1.75 | 0.41 | 0.76 |
| 14 d 37° C. | | | | | | |

*PO = impurity with one phosphorothioate moiety replaced by phosphate moiety (coeluting with 5′ N-1)
CNET = impurity with a cynoethyl-moiety added to one of the thymidine nucleotide
3′N-2 = impurity missing two 3′-terminal nucleotide
3′N-1 = impurity missing the 3′-terminal nucleotide
5′N-1 = impurity missing 5′-terminal nucleotide (coeluting with PO)
n.d. = not detected

The concentration of the samples were compared to the concentration of the Reference Samples (t=0 d). The data are summarized in Table 7. The results are considered to be adequate, when the concentration was between 95 and 105% of the concentration of the respective Reference Sample.

TABLE 7
UV-Analysis of OT-101 Solution of 10 μM
(61.43 μg/mL) in 0.9% NaCl

| Sample ID | A260 nm | Concentration (%) |
|---|---|---|
| 10 μM in saline, t = 0 d −20° C. | 0.5291 | 100.0 |
| 10 μM in saline, t = 7 d 5° C. | 0.5321 | 100.6 |
| 10 μM in saline, t = 14 d 5° C. | 0.5282 | 99.8 |
| 10 μM in saline, t = 7 d 37° C. | 0.5326 | 100.7 |
| 10 μM in saline, t = 14 d 37° C. | 0.5288 | 99.9 |

All UV spectra corresponded to the characteristic UV spectrum of OT-101 and the concentrations of all solutions were within a range of +0.8% of the concentration of the reference (t=0 d). This experiment demonstrated that the concentrations of a 10 μM (61.43 μg/mL) OT-101 solution in isotonic saline after storage at 5° C. and 37° C. for two weeks were substantially unchanged.

These experiments showed that based on the above RP-HPLC impurity levels, UV spectra and concentration profiles, the OT-101 antisense oligonucleotide solutions of 10 μM in NaCl at 5° C. and 37° C. were surprisingly stable for at least two weeks.

A further in-use stability study of OT-101 at 7.35 mg/mL and 25 mg/mL was performed. OT-101 solutions of concentrations 7.35 mg/mL and 25 mg/mL in 0.9% NaCl were checked for stability after storage at 5° C. and 37ºC for two weeks. The materials used for the experiments are shown in Table 8.

TABLE 8
Materials and drug solution

| Description | Manufacturer | Ref. No. | Lot. No. |
|---|---|---|---|
| AP 12009 250 mg | Thymoorgan, | — | 08L10AP12009 |
| | Germany | | |
| 0.9% NaCl Solution | Braun, Germany | 3820084 | 0214A191 |
| 6R Sample Vials | PharMediPack, | 05000613100 | 20091188 |
| | Germany | | |
| Fluorotec Telfon | West Phar., | 12414110/40 Grey | 1072036272 |
| Stoppers | USA | | |
| Alu Caps | PharMediPack, | 03500103000 | 0507010302 |
| | Germany | | |

The impurity profiles of the samples were determined by RP-HPLC and the concentrations were determined by UV-spectrometry. The impurities profiles of the samples by RP-HPLC are shown in Table 9.

TABLE 9
Reverse Phase HPLC Samples for 7.35 mg/mL and 25 mg/mL Solutions

| | AP | | | 5′N- | | Total Other |
|---|---|---|---|---|---|---|
| | 12009 | 3′N-2* | 3′N-1* | 1/PO* | CNET* | Imp |
| Acceptance Criteria | ≥85 | ≤0.6 | ≤3.4 | ≤7.6 | ≤5.2 | Report |
| (AP 12009 Drug Product) | | | | | | |
| Description of Sample | 95.13 | n.d. | 1.14 | 1.78 | 0.47 | 1.48 |
| 7.35 mg/mL, t = 0 d −20° C. | 95.12 | n.d. | 1.14 | 1.76 | 0.48 | 1.51 |
| 7.35 mg/mL, t = 7 d 5° C. | 95.13 | n.d. | 1.15 | 1.77 | 0.47 | 1.48 |
| 7.35 mg/mL, t = 14 d 5° C. | 94.79 | n.d. | 1.19 | 2.01 | 0.47 | 1.55 |
| 7.35 mg/mL, t = 7 d 37° C. | 94.57 | n.d. | 1.24 | 2.13 | 0.48 | 1.59 |
| 7.35 mg/mL, t = 14 d 37° C. | 95.08 | n.d. | 1.15 | 1.75 | 0.47 | 1.55 |
| 25 mg/mL, t = 0 d −20° C. | 95.04 | n.d. | 1.15 | 1.74 | 0.47 | 1.74 |
| 25 mg/mL, t = 7 d 5° C. | 95.05 | n.d. | 1.13 | 1.77 | 0.47 | 1.57 |
| 25 mg/mL, t = 14 d 5° C. | 94.81 | n.d. | 1.18 | 1.91 | 0.48 | 1.63 |
| 25 mg/mL, t = 7 d 37° C. | 94.62 | n.d. | 1.23 | 1.98 | 0.47 | 1.98 |
| 25 mg/mL, t = 14 d 37° C. | 95.13 | n.d. | 1.14 | 1.78 | 0.47 | 1.48 |

*PO = impurity with one phosphorothioate moiety replaced by phosphate moiety (coeluting with 5′ N-1)
CNET = impurity with a cynoethyl-moiety added to one of the thymidine nucleotide
3′N-2 = impurity missing two 3′-terminal nucleotide
3′N-1 = impurity missing the 3′-terminal nucleotide
5′N-1 = impurity missing 5′-terminal nucleotide (coeluting with PO)
n.d. = not detected

The impurity profile was adequate for the intended administration.

The concentrations of the samples were compared to the concentrations of the Reference Samples (t=0 d). The data are summarized in Table 10 and Table 11. The results were adequate, when the concentration was between 95 and 105% of the concentration of the respective Reference Sample.

TABLE 10
UV-Analysis of OT-101 Solution of 7.35 mg/mL in 0.9% NaCl Solution

| Sample ID | A₂₆₀ₙₘ | Concentration (%) |
|---|---|---|
| 7.35 mg/mL, t = 0 d @ −20° C. | 0.4441 | 100.00 |
| 7.35 mg/mL, t = 7 d @ 5° C. | 0.4516 | 101.69 |
| 7.35 mg/mL, t = 14 d @ 5° C. | 0.4534 | 102.09 |
| 7.35 mg/mL, t = 7 d @ 37° C. | 0.4525 | 101.89 |
| 7.35 mg/mL, t = 14 d @ 37° C. | 0.4519 | 101.76 |

TABLE 11
UV-Analysis of OT-101 Solution of 25 mg/mL in 0.9% NaCl

| Sample ID | A₂₆₀ₙₘ | Concentration (%) |
|---|---|---|
| 25 mg/mL, t = 0 d @ −20° C. | 0.4837 | 100.00 |
| 25 mg/mL, t = 7 d @ 5° C. | 0.4960 | 102.54 |
| 25 mg/mL, t = 14 d @ 5° C. | 0.4949 | 102.32 |
| 25 mg/mL, t = 7 d @ 37° C. | 0.5010 | 103.58 |
| 25 mg/mL, t = 14 d @ 37° C. | 0.4989 | 103.14 |

All UV spectra corresponded to the characteristic UV spectrum of OT-101 and the concentrations of all solutions were within a range of +3.58% of the concentration of the reference (t=0 d). This experiment demonstrated that the concentrations of 7.35 mg/mL and 25 mg/mL of OT-101 solution in isotonic saline after storage at 5° C. and 37° C. for two weeks are surprisingly stable and unchanged. Based on the above RP-HPLC impurity levels, UV spectra and concentration profiles, the OT-101 solutions of 7.35 mg/mL and 25 mg/mL in isotonic saline solution at 5° C. and 37° C. were surprisingly stable for at least two weeks.

The experiments set forth above further showed that a 15 mg/mL OT-101 Drug Solution in isotonic saline at a flow rate of 1 mL/h over a period of four days was surprisingly stable for the intended Drug Delivery System for IV Infusion.

The experiments set forth above further showed that a 10 μM (61.43 μg/mL) OT-101 Drug Solution in isotonic saline at a flow rate of 0.24 mL/h over a period of seven days was surprisingly stable for the intended Drug Delivery System for IV Infusion.

Example 5. New medical compositions, preparations, and methods discovered for inhibiting TGF-β using primary and alternative binding sites. This example demonstrates identification and use of new medical compositions, preparations, and methods which have been discovered for inhibiting TGF-β. New compositions, preparations, and methods were discovered using bioinformatic structure-based ligand design to identify and measure primary and alternative binding sites of TGF-β1.

Protein crystal structure for TGFβ1 was retrieved from protein data bank (https://www.rcsb.org/) with the accession code 3KFD. The protein was prepared by adding hydrogen atom, removing salts and ion. Missing side chains and loops were added. Finally, proteins were subjected to energy minimization to relax the coordinates. All other parameters were kept default. PocketFinder bioinformatic platform was used to detect primary and alternative binding sites of the protein target. The results were analyzed to identify the structure of binding sites and the orientations of residues neighboring a bound ligand.

A ligand structure based on artemisinin was used for docking calculations with the structure of TGFβ1. Before docking, the test structure was optimized to relax the coordinates. Pocket residues were selected to generate the grid before docking, and a grid was generated for each identified site. Docking of the artemisinin ligand structure was carried out in the generated grid for each target individually. Before docking all parameters were kept default. Ten poses were generated for the docked ligand at each site, and a single final pose was obtained as a result. Each docking output was scored and the ligand conformation determined. The nature and kind of binding interactions for the ligand were determined.

The three dimensional architecture of the protein was mainly composed of beta sheets and long flexible loops. The structure was not tightly packed, so that targeting with small molecules required extensive calculations. Small hydrophobic sub-pockets were formed into which small molecules such as artemisinin could be occupied with the polar side exposed to solvent. Solvent-exposed sites or pockets were detected for which solvent-accessible surface area of the protein was very high.

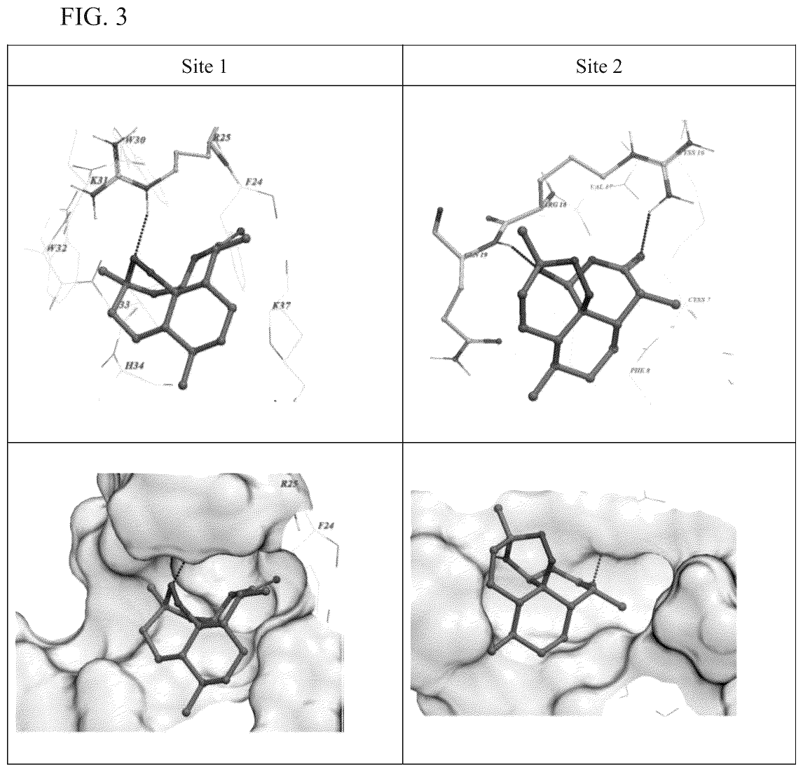

The results determined two sites for binding activity. As shown in FIG. 3, Site 1 included residues Phe24-Lys37 with a docking score of −1.230. Site 2 included residues Cys7-Gln 19 with a docking score of −6.01. Site 1 and Site 2 can be used for screening of molecules which will bind into these pockets to block TGF-β activity.

Site II indicated improved ligand sampling inside the pocket for improved binding. The binding interactions of the ligand were within hydrogen bonding distance, which confirmed enzyme-inhibiting activity. Moreover, polar groups of artemisinin occupied deep pocket orientations and confirmed enzyme-inhibiting activity. In particular, the results showed that the keto group of the artemisinin ligand formed a hydrogen bond with the side chain of ARG15. Further, the ether group of the ligand formed a hydrogen bond with the GLN19 backbone NH. A weak hydrophobic interaction was observed between the ligand and PHE8. The core of the pocket was solvent exposed. These structural features confirmed enzyme-inhibiting binding and activity.

New drug agent molecules or ligands which bind to Site 1 or Site 2 have been identified. In some embodiments, artemisinin and its derivatives are agent molecules or ligands which bind to Site 1 or Site 2 and inhibit a TGF-β target, which can include pharmaceutically-acceptable salts, salt polymorphs, esters, or isomers thereof.

In further embodiments, compounds or ligands comprising a small molecule or polypeptide that interacts with Site I of TGF-β comprising Trp30 and/or Site II of TGF-β comprising Arg15, Gln19, and Phe8, or a pharmaceutically-acceptable salt, salt polymorph, ester, or isomer thereof are agent molecules or ligands which bind to Site 1 or Site 2 and inhibit a TGF-β target.

In additional embodiments, polypeptides or peptide mimetics of Site I of TGF-β comprising residues Phe24-Lys37 and/or Site II of TGF-β comprising residues Cys7-Gln19, or a pharmaceutically-acceptable salt, salt polymorph, ester, or isomer thereof are agent molecules or ligands which bind to Site 1 or Site 2 and inhibit a TGF-β target.

In certain embodiments, an antibody or antibody fragment with affinity for Site I of TGF-β comprising residues Phe24-Lys37 and/or Site II of TGF-β comprising residues Cys7-Gln19 are agent molecules or ligands which bind to Site 1 or Site 2 and inhibit a TGF-β target.

In alternative embodiments, compounds comprising a sesquiterprene lactone or derivative thereof, or a pharmaceutically-acceptable salt, salt polymorph, ester, or isomer thereof are agent molecules or ligands which bind to Site 1 or Site 2 and inhibit a TGF-β target.

In further embodiments, compounds comprising three isoprenyl groups and one lactone ring, or derivatives thereof, or a pharmaceutically-acceptable salt, salt polymorph, ester, or isomer thereof are agent molecules or ligands which bind to Site 1 or Site 2 and inhibit a TGF-β target.

Example 6. Bioavailability and tolerance study of nasal formulations of apomorphine HCl. Objectives and endpoints: To determine the tolerance, safety, and pharmacokinetics of apomorphine HCl in healthy subjects. Safety and tolerance were assessed via adverse events and a nasal tolerance questionnaire. The pharmacokinetic parameters: Cmax, tmax, AUC0-180, t½, and kel were derived.

Methodology: Single center, single dose, open-label study to evaluate the safety, tolerability, and pharmacokinetics of intranasal apomorphine HCl at dosage levels ranging from 0.1 mg to 2.0 mg per 0.1 ml in healthy male subjects, and at dose levels of 0.1 mg to 0.75 mg in healthy female subjects.

Investigation of each dose level comprised one visit. Eligible subjects underwent a physical examination, nasal examination, and blood pressure, pulse rate, and respiration rate were recorded before dosing. Blood samples were drawn at 5, 10, 15, 20, 30, 45, 60, 90, 120 and 180 minutes after dosing and subjects were assessed for adverse events from dosing through to discharge (at approximately 3 hours after dosing). Blood pressure, pulse rate, and respiration rate were recorded 30 minutes after dosing, and at discharge. A nasal examination was also completed prior to discharge. Subjects were not permitted to eat during the study until after the 120-minute blood sample had been drawn. Hot liquids were prohibited for 90 minutes prior to dosing and for 240 minutes post dosing. There was a minimum washout of 3 days between doses.

Diagnosis and Main Criteria for Inclusion: Subjects recruited to the original protocol were healthy non-smoking males aged 18-45 years, inclusive. Protocol Amendment 5 extended the study to permit the entry of healthy, non-smoking female subjects aged 18-45 years. Subjects with nasal conditions likely to affect nasal absorption (such as chronic nosebleeds, allergic rhinitis, severe deviated nasal septum) were excluded from the study.

Drugs, Doses, and Regimens: At each treatment visit, male subjects received one dose of the following:
Apomorphine HCl nasal spray, 0.1 mg per 0.1 ml; lot #L/N 00015A
Apomorphine HCl nasal spray, 0.25 mg per 0.1 ml; lot #L/N 00013A
Apomorphine HCl nasal spray, 0.50 mg per 0.1 ml; lot #L/N 00014A
Apomorphine HCl nasal spray, 1.0 mg per 0.1 ml; lot #L/N 99049A
Apomorphine HCl nasal spray, 2.0 mg per 0.1 ml; lot #L/N 99049A
   At each treatment visit, female subjects received one dose of the following.
Apomorphine HCl nasal spray, 0.1 mg per 0.1 ml; lot #L/N 00018A
Apomorphine HCl nasal spray, 0.25 mg per 0.1 ml; lot #L/N 00019A
Apomorphine HCl nasal spray, 0.50 mg per 0.1 ml; lot #L/N 00020A
Apomorphine HCl nasal spray, 0.75 mg per 0.1 ml; lot #L/N 99023A.

Statistical Methods: The pharmacokinetic parameters were derived using PKAnalyst Software. A linear fitting operation and least squares minimization algorithm were used to fit the data to the one-compartment, first order input, first order output model.

Results: In total, 32 healthy male volunteers received 75 doses of study treatment. Subjects were permitted to enter more than one dose level; 16 subjects received only one dose level; five subjects received two dose levels; two subjects received three and two subjects received four dose levels, seven subjects received all five dose levels.

However, due to problems with pharmacokinetic analysis of some samples at the 1 and 2 mg dose levels, only 47 doses were analyzed for pharmacokinetics (12 at the 0.1 and 0.25 mg levels, 11 at the 0.50 mg level, and 6 each at the 1.0 and 2.0 mg levels).

In addition, 14 healthy female subjects received 48 doses of study treatment; 10 received all four dose levels, two received only three dose levels, and two received only one dose level, giving 12 subjects per dose level.

Pharmacokinetic Results: In males, there was a dose dependent increase in plasma Cmax with tmax obtained within approximately 20 minutes; a 50% decrease in plasma levels was noted at 40 to 60 minutes after dosing. There was relatively low subject-to-subject variability in Cmax (coefficient of variation [CV] 24-63%) and AUC (CV 26-60%). Table 12 summarizes the pharmacokinetic data for males.

TABLE 12
Single Dose Pharmacokinetics of Intranasal
Apomorphine in Healthy Males

| Apomorphine | 0.10 | 0.25 | 0.50 | 1.0 | 2.0 |
|---|---|---|---|---|---|
| dose (mg) | n = 12 | n = 12 | n = 11 | n = 6 | n = 6 |
| Cmax (ng/ml) | 0.063 | 0.189 | 0.554 | 1.194 | 2.720 |
| Tmax (min) | 16.38 | 17.95 | 21.25 | 20.64 | 16.23 |
| AUC₀₋₁₈₀ (ng/ml · min) | 2.295 | 7.975 | 29.63 | 70.65 | 128.2 |
| t_{1/2} (min) | 11.40 | 12.43 | 14.73 | 14.31 | 11.25 |
| Ke1 (ng · min/ml) | 0.061 | 0.056 | 0.047 | 0.048 | 0.062 |

In females, apomorphine was detectable in the blood within 5 minutes, and subjects achieved maximum levels within 22 to 28 minutes of dosing. The tmax was independent of dose. Cmax values ranged between 0.031 and 0.479 ng/ml. Table 13 summarizes the pharmacokinetic data for females.

TABLE 13
Single Dose Pharmacokinetics of Intranasal
Apomorphine in Healthy Females

| Apomorphine | 0.10 | 0.25 | 0.50 | 0.75 |
|---|---|---|---|---|
| dose (mg) | n = 12 | n = 12 | n = 12 | n = 12 |
| Cmax (ng/ml) | 0.031 | 0.172 | 0.294 | 0.479 |
| Tmax (min) | 26.85 | 24.53 | 28.95 | 22.10 |
| AUC₀₋₁₈₀ (ng/ml · min) | 0.733 | 11.96 | 22.97 | 27.43 |
| t_{1/2} (min) | 18.65 | 24.25 | 23.95 | 15.33 |
| Ke1 (ng · min/ml) | 0.037 | 0.029 | 0.029 | 0.045 |

Pharmacokinetic studies with an intranasal formulation of apomorphine have shown that:

The maximum plasma concentration of apomorphine was obtained more quickly with the intranasal formulation compared to the published values for the sublingual formulation (tmax values of 15-20 minutes and 45 minutes respectively). Cmax was approximately four times higher with the intranasal dose compared to the sublingual dose (2.7 ng/ml and 0.7 ng/ml respectively for a 2 mg dose).

The exposure at the 2 mg intranasal dose in males was approximately twice as high as the value reported for the same dose administered sublingually (AUC0-180 of 2.1 ng·h/ml for the intranasal formulation compared to AUC0-∞ of 1.23 ng·h/ml for the sublingual formulation).

Intranasally administered apomorphine was also cleared from the body much more rapidly than reported for the sublingual formulation. The reported t½ values are 11-15 minutes for the intranasal formulation compared with 2-4 hours for the sublingual formulation.

It therefore appears that the intranasal route of administration could be a more efficient route of drug delivery than the sublingual route, with more rapid delivery of maximum plasma concentrations. SL Apomorphine (Uprima) apomorphine was rapidly absorbed from the sublingual cavity and can be detected in plasma within 10 minutes after placing the tablet under the tongue. Peak plasma concentrations are attained in about 40-60 minutes. Increasing dosage strengths of Uprima sublingual tablets provide dose-proportional increases in Cmax and AUC. The bioavailability of apomorphine from sublingual tablets, relative to subcutaneous administration, was approximately 17-18%. Comparison to sublingual apomorphine these are the findings:

AL-101 (IN) was faster: sublingual (SL) tablet dissolution itself may be limiting.

AL-101 (IN) was more efficient: IN higher Cmax was related to rapid uptake and good absorption; IN lower AUC was related to total absorption.

AL-101 (IN) was less variable: “Safety may be difficult to predict (for SL formulation) based on dose due to variability in Cmax.

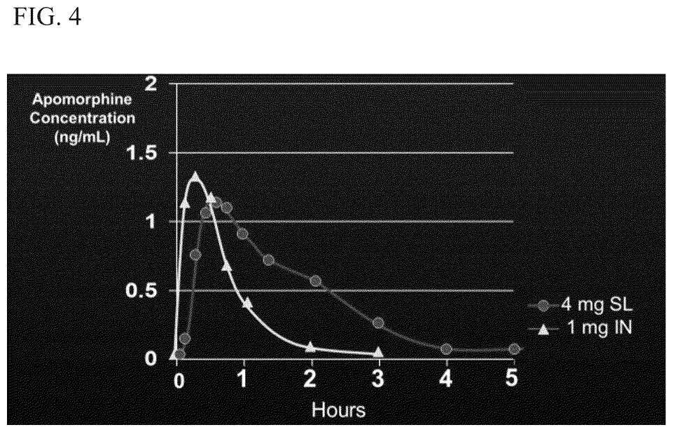

These data are shown graphically in FIG. 4.

Example 7. Safety and efficacy of nasal formulations of apomorphine HCl. Title: A pilot, double-blind, double dummy, controlled, crossover study to assess the tolerance, safety and potential efficacy of nasal formulations of apomorphine HCl versus placebo and Viagra□ in subjects with erectile dysfunction principally of psychogenic origin.

Objectives and endpoints: To assess the nasal tolerance, adverse drug reaction profile, and efficacy of apomorphine HCl, in doses ranging from 0.25 mg to 1.0 mg per 0.1 ml, as compared to placebo and Viagra in male subjects with erectile dysfunction principally of psychogenic origin. The primary efficacy parameter was each subject's assessment of the quality of the erection (graded on a 4-point scale). Secondary endpoints included frequency of erection, time from dosing to erection, duration of erection, and efficacy index (EI) for subjects achieving erections. Safety was primarily assessed via adverse events but cardiovascular effects were also investigated by monitoring blood pressure, heart rates, and percent oxygen saturation, prior to and following dosing. The integrity of the nasal mucosa of both nostrils was also evaluated prior to dosing, in the event of nasal symptoms, and at the end of each treatment visit.

Methodology: Single center, single dose, double-blind, double dummy, controlled crossover study to evaluate the safety, tolerability, and efficacy of intranasal apomorphine HCl at dosage levels ranging from 0.25 mg to 1.0 mg per 0.1 ml as compared to placebo and Viagra□ in male subjects with erectile dysfunction principally of psychogenic origin. Subjects made a total of six visits to the site, an initial screening and qualification visit, three treatment visits in Part 1, and two treatment visits in Part 2. Eligible subjects were randomized to a treatment sequence for Part I after the initial screening visit. Approximately 2 months later, subjects were randomized to a treatment sequence for Part 2.

At each treatment visit, a single dose of study treatment was administered. Efficacy was measured by means of ED questionnaires, which were completed by each subject. Following dosing, all subjects viewed sexually explicit videotapes and magazines for approximately 60 minutes. At the end of this period, the questionnaires were completed and patients were asked to rate the quality of the erection using a 4-point scale. The primary efficacy variable was the subject's global rating of erection, measured on a scale of 1 to 4: 1=increase in size but not hard; 2=hard, but not hard enough for vaginal penetration; 3=hard enough for vaginal penetration (but not completely hard); 4=completely hard.

Safety was measured by monitoring the subjects' blood pressure, heart rate and percent oxygen saturation (by pulse oximetry) before and after dosing (approximately 90 minutes after dosing commenced). In addition, the integrity of the subject's nasal mucosa was assessed prior to, and at the end of treatment. Information on adverse events was collected throughout the period of the study.

The duration of the entire study ranged between 3-4 months per subject. The minimum time between any two treatments was 24 hours. The first part of the study was conducted over a 1-month period, and Part 2 was conducted approximately 2 months later.

Diagnosis and Main Criteria for Inclusion: Subjects were heterosexual males aged 18-65 years, inclusive, with a self-reported history of erectile dysfunction of >6 months duration, due to non-organic etiologies (confirmed by medical records or diagnosis by intracavernosal injection). Subjects were in good overall health, without clinically significant laboratory profiles, with normal nasal mucosa in the nostril used for administration of the test products.

Subjects with nasal conditions likely to affect nasal absorption (such as chronic nosebleeds, allergic rhinitis, severe deviated nasal septum) were excluded from the study. Also excluded were subjects with clinically significant cardiovascular or respiratory diseases, specifically those receiving organic nitrates or nitric oxide donors as concomitant medications.

Drugs, Doses, and Regimens: At each treatment visit, subjects received one dose of study treatment. Subjects received the following in a randomized sequence.
Apomorphine HCl nasal spray, 1.0 mg in 0.1 ml
Viagra 50 mg tablets
Placebo nasal spray formulation to match the apomorphine HCl test products.

Results: Of 24 subjects screened, 21 were enrolled and completed the first phase of the study. Of these 21 subjects, 18 went on to complete the second phase of the study.

Efficacy Results: Using Global Self Assessment Scores (grade 3 or 4), 39% efficacy was observed in the placebo group. The Viagra□ group demonstrated efficacy of 67%. Efficacy of the intranasal apomorphine groups ranged from 72 to 82%. The difference between apomorphine 0.5 mg and placebo was statistically significant. The results showed that 60 to 70% of subjects treated with nasal apomorphine achieved a satisfactory erection (as reported by the subject), compared to around 30% in the placebo and Viagra® groups; the difference between the apomorphine 0.5 mg and placebo group was statistically significant (p=0.03). Time of onset and duration of erection were similar across all five groups.

Conclusions: This study demonstrated no statistically significant difference between the effectiveness of apomorphine HCl at doses of 0.25, 0.50, and 1.0 mg, Viagra 50 mg, and placebo in initiating erections. The adverse event profiles of the treatments were similar and there were no serious adverse events during the study. No clinically significant changes in cardiovascular parameters were detected although small decreases in heart rate were detected after each of the apomorphine doses, and after placebo.

### Study 2.

Title: A double-blind, fixed dose at home proof of concept study to assess the safety and efficacy of apomorphine HCl delivered as a nasal spray preparation for the treatment of erectile dysfunction of psychogenic or organic origin.

Objectives and endpoints: To assess safety and efficacy of apomorphine HCl in an at-home setting in patients with erectile dysfunction of psychogenic or organic origin. Primary efficacy was based on responses to SEP Question 2 (“Were you able to achieve at least some erection?”) and Question 3 (“Were you able to insert your penis into your partner's vagina?”). Safety was primarily assessed via adverse events; nasal mucosa examinations were performed at the clinic visits, and vital signs were recorded.

Methodology: This was a randomized, multicenter, double-blind, fixed dose study in males with ED of all etiologies and severities. Demographic analysis at baseline showed that 50% of those participating in the study had ED of psychogenic origin, 26% of mixed organic origin and 24% of diabetic origin.

Following screening, patients were randomly allocated to four groups; placebo, 0.25, 0.5 or 1.0 mg apomorphine. Treatment consisted of up to 18 doses, Study treatment was taken 15-20 minutes before sexual intercourse but not more than once daily. Patients completed the sexual encounter profile (SEP) at home diary each time they took study treatment and attempted intercourse. Patients returned to the clinic after every sixth dose, during the clinic visits they completed the validated ED questionnaire (the international index of erectile function [IIEF] score). Additionally, a global efficacy question was answered at the end of the study.

Diagnosis and Main Criteria for Inclusion: Subjects were heterosexual males aged 18-75 years, inclusive, with erectile dysfunction of psychogenic or organic origin of >3 months duration.

Drugs, Doses, and Regimens: Subjects were randomized to one of the following treatments:
Placebo nasal spray
Apomorphine HCl nasal spray, 0.25 mg in 0.1 ml
Apomorphine HCl nasal spray, 0.50 mg in 0.1 ml
Apomorphine HCl nasal spray, 1.0 mg in 0.1 ml.

Statistical Methods: Analysis of covariance (ANCOVA), and logistic regression with 95% confidence intervals, as appropriate were used to compare the treatment groups. Statistical tests were 2-sided and made at the 5% significance level.

Results: Of 246 patients screened, 184 were enrolled and 125 completed the study.

Efficacy Results: Compared with placebo, more patients receiving apomorphine HCl were able to achieve some erection and insert their penis into their partner's vagina. For example, the success rate for achieving vaginal penetration was 73% and 82% for patients receiving the 0.5 and 1.0 mg dose of apomorphine HCl compared to 36% for those receiving placebo, shown in Table 14.

TABLE 14
Efficacy Results

| | 0.25 mg | 0.5 mg | 1.0 mg | Placebo |
|---|---|---|---|---|
| Apomorphine dose group | (n = 12) | (n = 55) | (n = 39) | (n = 57) |
| % patients with some erection | 81.5 | 89.4* | 90.6* | 68.9 |
| achieved (SEP question 2) | | | | |
| % patients whose erection was | 68.8* | 73.4* | 81.5* | 35.5* |
| sufficient for vaginal penetration | | | | |
| (SEP question 3) | | | | |

Per protocol population, doses 7-18
*p < 0.05 and **p < 0.0001 for comparison of apomorphine versus placebo

Conclusions: The results indicated that intranasal apomorphine offers a safe, well tolerated and efficacious treatment (particularly at the 0.5 mg and 1.0 mg dose levels) for erectile dysfunction of psychogenic or organic origin.

A 6-month open-label extension in approximately 30 patients was added to this study. Subjects on placebo were titrated to 0.5 mg of active drug, subjects on 0.25 mg were titrated to 1.0 mg of active drug, subjects on 0.5 mg were titrated to 1.0 mg or remained on 0.5 mg of apomorphine, and subjects on 1.0 mg remained at that dose. STUDY 3.

Title: A pilot phase II randomized, double-blind, placebo-controlled, parallel design study of the efficacy and safety of at home, on demand dosing of intranasal apomorphine HCl in pre-menopausal patients with acquired female sexual dysfunction.

Objectives and Endpoints: The objective of this study was to evaluate the safety and efficacy of at-home dosing of nasal apomorphine at 0.5 mg compared to placebo, in the treatment of pre-menopausal women on oral contraceptives, who have female sexual arousal disorder.

Methodology: The trial was designed as a pilot, randomized, double blinded, placebo-controlled, parallel group study. Subjects are enrolled in a 4-week pre-treatment period followed by and 12-week in-home treatment period. Subjects will complete pre and post treatment questionnaires regarding sexual function and will also complete a sexual event log after each in-home dose of study medication.

Diagnosis and Main Criteria for Inclusion: Pre-menopausal women taking oral contraceptives who have a diagnosis of acquired female sexual arousal disorder.

### Drugs, Doses, and Regimens:

Apomorphine 0.5 mg nasal spray
Placebo nasal spray.

Subjects will be randomized with a 2:1 ratio of active to placebo. They are instructed to take not more than 1 dose in 24 hours. They are dispensed 11 doses for the first 4-week treatment period and 12 doses for subsequent 4-week treatment period.

Results: apomorphine was effective against female sexual arousal disorder.

Summary of Efficacy and Safety is shown in Table 15.
Nasal apomorphine has a low Adverse Event profile
Over 200 patients (2,200 doses) have participated in clinical trials for intranasal apomorphine (including geriatric patients up to 78 years old)
Very low incidence of nausea
To date no incidences of vomiting, syncope or hypotension in patients
The data are presented below
The preferential delivery to CNS reduced the side effects associated with apomorphine
The side effect of intranasal apomorphine was superior to Viagra and Cialis.

TABLE 15
Summary of Efficacy

| Adverse | Apomorphine | | | Apomorphine |
|---|---|---|---|---|
| Event | IN | Viagra | Cialis | SL |
| Headache | 0.8% | 16% | 13.9% | 6.5% |
| Nausea | 0.8% | <2% | ? | 22.2% |
| Dizziness | 3.3% | 2% | 6.2% | 14.5% |
| Flushing | 0% | 10% | 4.2% | 6.5% |
| Dyspepsia | 0% | 7% | 7.7% | ? |
| Vomiting | 0% | <2%, Not 0% | ? | 4.3% |
| Hypotension | 0% | <2%, Not 0% | ? | 6% |
| Syncope | 0% | 0.14% | ? | 2% |

Example 8. Evaluation of cerebrospinal fluid (CSF) apomorphine levels following intranasal and sublingual administration. Objectives and endpoints: To compare CSF levels of apomorphine in healthy males following intranasal and sublingual administration, and to compare CSF apomorphine levels with plasma levels.

Methodology: This was an open, crossover comparison of two single doses of apomorphine administered to each of the six study arms. A washout of at least 3 days was to elapse between doses.

Following dosing, subjects underwent lumbar puncture. Lumbar puncture was performed at 15, 20, and 30 minutes post dosing (a third of the subjects in each arm were to be sampled at each of these times). Blood samples were drawn at 0, 5, 10, 20, 30, 60, and 120 minutes after dosing. Subjects were assessed for adverse events from dosing through to discharge (at approximately 4 hours after dosing). Nasal examination and vital signs were also recorded at intervals during the study. Subjects were followed-up 24-48 hours after discharge by telephone.

Diagnosis and Main Criteria for Inclusion: Healthy males aged 18-40 years, inclusive, who are non-smokers.

Drugs, Doses, and Regimens: Subjects were administered intranasal and sublingual apomorphine as follows:

| | Arm 1 | Arm 2 | Arm 3 | Arm 4 | Arm 5 | Arm 6 |
|---|---|---|---|---|---|---|
| Nasal | 0.25 mg | 0.5 mg | 1.0 mg | 0.25 mg | 0.5 mg | 1.0 mg |
| Sublingual | 2 mg | 2 mg | 2 mg | 3 mg | 3 mg | 3 mg |

Results: (a) The subcutaneous formulation produced 2.5-4.3% levels in the CSF compared to plasma. (b) The intranasal formulation produces 26.7-44.1% levels in the CSF relative to plasma. (c) The intranasal formulation provides CSF levels that are four (4) standard deviations higher than subcutaneous formulation. (d) The direct administration to the CSF through intranasal route resulted in preferential accumulation in CSF suggesting that there is little leakage from CSF into systemic circulation suggesting that direct administration either through lumbar puncture or through Ommaya reservoir would localize apomorphine to the central nervous system. (e) The intranasal route would be preferred for delivery of the apomorphine to the CSF with minimum systemic exposure to avoid side effects associated with systemic apomorphine. Further to this point—intrathecal administration either through lumbar puncture or through Ommaya reservoir especially for severe neurological disease would be desirable.

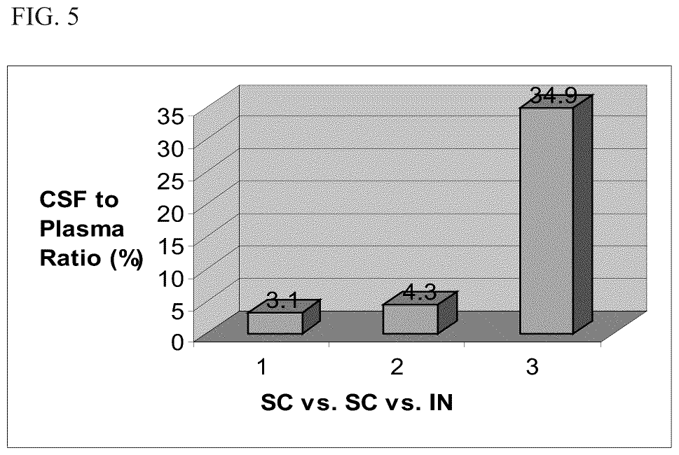

The results of this example are shown in FIG. 5.

Example 9. Evaluation of the efficacy and safety of two doses of OT-101 in adult patients with recurrent high-grade glioma. Study G004 was a multi-national, multi-center, open-label, active-controlled, randomized parallel-group dose-finding study to evaluate the efficacy and safety of two doses of OT-101 in adult patients with recurrent high-grade glioma, administered intratumorally as continuous high-flow microperfusion over a 7-day period every other week (NCT00431561). In addition, efficacy and safety of the 2 doses of OT-101 were compared to standard chemotherapy (TMZ or PCV). Ninety-eight (98) patients (AA: 30; GBM: 68) were randomized to one of the 2 treatment arms (intent-to-treat population [ITT]) of OT-101 representing 2 different dose cohorts, namely 2.5 mg/cycle (N=48) and 19.8 mg/cycle (N=50), respectively.

In our phase 2 clinical trial (NCT00431561), OT-101 was administered via continuous intracranial infusion over 7 days to 89 adults (62 GBM and 27 AA patients) with R/R high grade gliomas via intracranial delivery with an intratumoral catheter using a CED system. The intended minimum number of the 7-day OT-101 cycles was 4 and the maximum allowed number of 7-day OT-101 cycles was 11.

In comparison to the control arm (TMZ), OT-101 treated patients have 3X the level of psychiatric changes with 32% of treated pts with aggression (5%), agitation (5%), anxiety (5%), confusion (12%), insomnia (5%), mood changes (2%).

What is claimed is:

1. A therapeutic composition for treating a neurological disease or disorder comprising a therapeutically effective amount of apomorphine, an apomorphine pro-drug, or a pharmaceutically acceptable salt or ester thereof.

2. The therapeutic composition of claim 1, wherein the neurological disease or disorder is Parkinson's Disease, Alzheimer's Disease, male or female sexual dysfunction, or excessive daytime sleepiness.

3. The therapeutic composition of claim 1, wherein the neurological disease or disorder is early or late Parkinson's Disease.

4. The therapeutic composition of claim 1, wherein the apomorphine is Apomorphine Hydrochloride.

5. The therapeutic composition of claim 1, wherein the composition is suitable for intrathecal injection, infusion, or intranasal use.

6. The therapeutic composition of claim 1, wherein the composition is an intranasal powder formulation.

7. The therapeutic composition of claim 1, wherein the composition is an aqueous or non-aqueous formulation comprising any one or more of a pH buffer, a thickening agent, a humectant, a preservative, and one or more pharmaceutical excipients.

8. The therapeutic composition of claim 1, wherein the composition is an aqueous solution of gels, an aqueous suspension, an aqueous liposomal dispersion, an aqueous emulsion, an aqueous microemulsion, or a combination thereof.

9. The therapeutic composition of claim 1, wherein the composition is an aqueous solution having a drug concentration of 5 mg or 10 mg per mL of solution.

10. The therapeutic composition of any of claims 1-9, wherein the composition comprises a buffer selected from acetate, citrate, prolamine, carbonate, phosphate, and combinations thereof.

11. The therapeutic composition of any of claims 1-9, wherein the composition comprises a thickening agent selected from methyl cellulose, xanthan gum, carboxymethyl cellulose, hydroxypropyl cellulose, carbomer, polyvinyl alcohol, alginates, acacia, chitosan, and combinations thereof.

12. The therapeutic composition of any of claims 1-9, wherein the composition comprises a humectant selected from sorbitol, glycerol, mineral oil, vegetable oil, and combinations thereof.

13. The therapeutic composition of any of claims 1-9, wherein the composition comprises a bio-adhesive excipient.

14. The therapeutic composition of any of claims 1-9, wherein the composition comprises any one or more of glycerin, glycol, propylene glycol, polyethylene glycol, polyethylene glycol 400, ascorbic acid, sodium ascorbate, edetate disodium, and sodium metabisulfite.

15. The therapeutic composition of any of claims 1-9, wherein the apomorphine is dispersed to improve solubility.

16. The therapeutic composition of any of claims 1-9, wherein the composition is active within 15 to 60 minutes.

17. The therapeutic composition of any of claims 1-9, comprising an intranasal dosage form of 0.5 mg or 1 mg per actuation at 0.1 mL per actuation.

18. The therapeutic composition of any of claims 1-9, comprising an intranasal formulation comprising one or more of an antioxidant, an antimicrobial, a chelating agent, a preservative, and combinations thereof.

19. The therapeutic composition of any of claims 1-9, comprising an intranasal formulation flushed with oxygen and nitrogen.

20. The therapeutic composition of any of claims 1-9, comprising an intranasal formulation with a pH of 3.4.

21. The therapeutic composition of any of claims 1-9, comprising a stable intranasal formulation after 3 months at 40° C./60% RH, or 24 months at 25° C./60% RH.

22. The therapeutic composition of any of claims 1-9, wherein the composition is pharmaceutically tolerable with reduced adverse or side effects.

23. A use of a therapeutic composition of any of claims 1-22 for treating or ameliorating the symptoms of a neurological disease or disorder in a human subject.

24. The use of claim 23, wherein the neurological disease or disorder is early or late Parkinson's Disease, Alzheimer's Disease, or male or female sexual dysfunction.

25. A use of a therapeutic composition of any of claims 1-22 in the preparation of a medicament for treating or ameliorating the symptoms of a neurological disease or disorder in a human subject.

26. The use of claim 25, wherein the neurological disease or disorder is early or late Parkinson's Disease, Alzheimer's Disease, or male or female sexual dysfunction.

27. A use of a therapeutic composition of any of claims 1-22 for treating or ameliorating the symptoms of a neurological disease or disorder in a human subject, wherein the use of the composition is combined with a standard of care treatment for the disease or disorder.

28. The use of claim 27, wherein the neurological disease or disorder is early or late Parkinson's Disease, Alzheimer's Disease, or male or female sexual dysfunction.

29. A use of a therapeutic composition of any of claims 1-22 for treating or ameliorating the symptoms of a neurological disease in a human or animal body.

30. The use of claim 29, wherein the neurological disease or disorder is early or late Parkinson's Disease, Alzheimer's Disease, or male or female sexual dysfunction.

31. A method for treating or ameliorating a symptom of a neurological disease or disorder, the method comprising administering the composition of any of claims 1-22.

32. The method of claim 31, wherein the neurological disease or disorder is early or late Parkinson's Disease.

33. The method of claim 31, wherein the administration is intranasal.

34. A therapeutic composition for treating a neurological disease or disorder comprising a therapeutically effective amount of an agent for inhibiting or suppressing expression of TGF-β.

35. The therapeutic composition of claim 34, wherein the neurological disease or disorder is Parkinson's Disease, Alzheimer's Disease, male or female sexual dysfunction, or excessive daytime sleepiness.

36. The therapeutic composition of claim 34, wherein the neurological disease or disorder is early or late Parkinson's Disease.

37. The therapeutic composition of claim 34, comprising any one or more pharmaceutically acceptable excipients selected from diluents, stabilizers, disintegrants and anticaking agents.

38. The therapeutic composition of claim 34, comprising any one or more excipients selected from microcrystalline cellulose, polysorbate 80, crospovidone, croscarmellose sodium, and magnesium stearate.

39. The therapeutic composition of claim 34, wherein the composition is suitable for use by intrathecal injection or infusion.

40. The therapeutic composition of any of claims 34-39, wherein the composition is pharmaceutically tolerable with reduced adverse or side effects.

41. The therapeutic composition of any of claims 34-39, wherein the agent for inhibiting or suppressing expression of TGF-β is an antisense oligonucleotide or inhibitor specific for TGF-β1, TGF-β2, or TGF-β3.

42. The therapeutic composition of any of claims 34-39, wherein the agent for inhibiting or suppressing expression of TGF-β is selected from TGF-β2-specific antisense oligonucleotides SEQ ID NOs:1-9:
SEQ ID NO:1, gtaggtaaaa acctaatat
SEQ ID NO:2, gttcgtttag agaacagatc
SEQ ID NO:3, taaagttcgt ttagagaaca g
SEQ ID NO:4, agccctgtat acgac
SEQ ID NO:5, gtaggtaaaa acctaatat
SEQ ID NO:6, cgtttagaga acagatctac
SEQ ID NO:7, cattgtagat gtcaaaagcc
SEQ ID NO:8, ctccctcatg gtggcagttg a
SEQ ID NO:9, cggcatgtct attttgta,
chemically-modified variants thereof, an artemisinin extract, and a pharmaceutically-acceptable salt, salt polymorph, ester, or isomer thereof, and any combination thereof.

43. The therapeutic composition of any of claims 34-42, wherein the agent for inhibiting or suppressing expression of TGF-β is an artemisinin formulation, comprising 90-95% pure artemisinin extract, or a pharmaceutically-acceptable salt, salt polymorph, ester, or isomer thereof, and one or more pharmaceutically acceptable excipients.

44. The therapeutic composition of any of claims 34-42, comprising a carrier comprising sterile water for injection, saline, isotonic saline, or a combination thereof.

45. The therapeutic composition of any of claims 34-42, wherein the composition is substantially free of excipients.

46. The therapeutic composition of any of claims 34-42, wherein the composition is stable for at least 14 days in carrier at 37° C.

47. The therapeutic composition of any of claims 34-42, wherein the composition is reconstituted from a lyophilized powder of the composition.

48. A use of a therapeutic composition of any of claims 34-42 in the preparation of a medicament for treating or ameliorating the symptoms of a neurological disease or disorder in a human subject.

49. The use of claim 48, wherein the neurological disease or disorder is early or late Parkinson's Disease, Alzheimer's Disease, or male or female sexual dysfunction.

50. A use of a therapeutic composition of any of claims 34-42 for treating or ameliorating the symptoms of a neurological disease or disorder in a human subject, wherein the use of the composition is combined with a standard of care treatment for the disease or disorder.

51. The use of claim 50, wherein the standard of care comprises any one or more additional medicaments comprising anti-inflammatoires, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

52. The use of claim 50, wherein the neurological disease or disorder is early or late Parkinson's Disease, Alzheimer's Disease, or male or female sexual dysfunction.

53. A use of a therapeutic composition of any of claims 34-42 for treating or ameliorating the symptoms of a neurological disease in a human or animal body.

54. The use of claim 53, wherein the neurological disease or disorder is early or late Parkinson's Disease, Alzheimer's Disease, or male or female sexual dysfunction.

55. A method for treating or ameliorating a symptom of a neurological disease or disorder, the method comprising administering the composition of any of claims 34-42.

56. The method of claim 55, wherein the neurological disease or disorder is early or late Parkinson's Disease.

57. The method of claim 55, wherein the administration is intrathecal injection, infusion, or direct intracranial administration.

58. A therapy for treating a neurological disease or disorder in a subject in need, the therapy comprising a combination of:
a therapeutically effective amount of an agent for inhibiting or suppressing expression of TGF-β; and
a therapeutically effective amount of apomorphine, an apomorphine pro-drug, or a pharmaceutically acceptable salt or ester thereof.

59. The therapy of claim 58, wherein the neurological disease or disorder is Parkinson's Disease, Alzheimer's Disease, male or female sexual dysfunction, or excessive daytime sleepiness.

60. The therapy of claim 58, wherein the neurological disease or disorder is early or late Parkinson's Disease.

61. The therapy of claim 58, wherein the apomorphine is Apomorphine Hydrochloride.

62. The therapy of claim 58, wherein the agent for inhibiting or suppressing expression of TGF-β comprises any one or more pharmaceutically acceptable excipients selected from diluents, stabilizers, disintegrants and anticaking agents.

63. The therapy of claim 58, wherein the agent for inhibiting or suppressing expression of TGF-β comprises any one or more excipients selected from microcrystalline cellulose, polysorbate 80, crospovidone, croscarmellose sodium, and magnesium stearate.

64. The therapy of claim 58, wherein the agent for inhibiting or suppressing expression of TGF-β is administered by intrathecal injection, infusion, or direct intracranial administration.

65. The therapy of claim 58, wherein the agent for inhibiting or suppressing expression of TGF-β is an antisense oligonucleotide or inhibitor specific for TGF-β1, TGF-β2, or TGF-β3.

66. The therapy of claim 58, wherein the agent for inhibiting or suppressing expression of TGF-β is selected from TGF-β2-specific antisense oligonucleotides SEQ ID NOs:1-9:
SEQ ID NO: 1
gtaggtaaaa acctaatat,
SEQ ID NO: 2
gttcgtttag agaacagatc,
SEQ ID NO: 3
taaagttcgt ttagagaaca g,
SEQ ID NO: 4
agccctgtat acgac,
SEQ ID NO: 5
gtaggtaaaa acctaatat,
SEQ ID NO: 6
cgtttagaga acagatctac,
SEQ ID NO: 7
cattgtagat gtcaaaagcc,
SEQ ID NO: 8
ctccctcatg gtggcagttg a,
SEQ ID NO: 9
cggcatgtct attttgta,,
chemically-modified variants thereof, an artemisinin extract, and a pharmaceutically-acceptable salt, salt polymorph, ester, or isomer thereof, and any combination thereof.

67. The therapy of claim 66, wherein the artemisinin is 90-95% pure artemisinin extract, or a pharmaceutically-acceptable salt, salt polymorph, ester, or isomer thereof, and one or more pharmaceutically acceptable excipients.

68. The therapy of claim 58, wherein the apomorphine is the therapeutic composition of any of claims 1-22.

69. The therapy of claim 58, wherein the agent for inhibiting or suppressing expression of TGF-β is the therapeutic composition of any of claims 34-39.

70. The therapy of any of claims 58-67, wherein the agent for inhibiting or suppressing expression of TGF-β comprises a carrier comprising sterile water for injection, saline, isotonic saline, or a combination thereof.

71. The therapy of any of claims 58-67, wherein the agent for inhibiting or suppressing expression of TGF-β is substantially free of excipients.

72. The therapy of any of claims 58-67, wherein the agent for inhibiting or suppressing expression of TGF-β is administered by intrathecal injection, infusion, or direct intracranial administration.

73. The therapy of any of claims 58-67, wherein the agent for inhibiting or suppressing expression of TGF-β is pharmaceutically tolerable with reduced adverse or side effects.

74. The therapy of any of claims 58-67, wherein the agent for inhibiting or suppressing expression of TGF-β is stable for at least 14 days in carrier at 37° C.

75. The therapy of any of claims 58-67, wherein the agent for inhibiting or suppressing expression of TGF-β is reconstituted from a lyophilized powder of the composition.

76. The therapy of any of claims 58-67, wherein the therapy comprises use of the agents with a standard of care treatment for the disease or disorder.

77. The therapy of claim 76, wherein the standard of care comprises any one or more additional medicaments comprising anti-inflammatoires, anti-inflammatory steroids, piperiquine, pyronaridine, curcumin, frankincense, Remdesivir, Sompraz D, Zifi CV/Zac D, CCM, Broclear, Budamate, Rapitus, Montek LC, low molecular weight heparine, prednisolone, Paracetamol, Vitamin B complex, Vitamin C, Pantoprozol, Doxycycline, Ivermectin, Zinc, Foracort Rotacaps inhalation, Injection Ceftriaxone, Tab Paracetamol, Injection Fragmin, Tablet Covifor, Azithromycin, Injection Dexamethasone, Injection Odndansetron, Tablet Multivitamin, Tablet Ascorbic Acid, Tablet Calcium Carbonate, and Tablet Zinc Sulfate.

78. The therapy of any of claims 58-67, wherein the agents are administered concurrently, simultaneously, sequentially, or separately.

79. The therapy of any of claims 58-67, wherein the apomorphine ingredient is administered alone in an early stage of the neurological disease or disorder, and wherein both the apomorphine ingredient and the agent for inhibiting or suppressing expression of TGF-β are administered in a later stage of the neurological disease or disorder.

80. The therapy of any of claims 58-67, wherein the apomorphine ingredient is administered alone in an early stage of the neurological disease or disorder when the subject does not have an elevated level of TGF-β, and wherein both the apomorphine ingredient and the agent for inhibiting or suppressing expression of TGF-β are administered in a later stage of the neurological disease or disorder when the subject has an elevated level of TGF-β.
